SEQUENCE LISTING (8 sequences)

SEQ ID NO: 1 - DNA, 61 bp, Homo sapiens
aagtaatggtatattaaatttggaatatagmgaaaacaatgacccataatgtcatgataaa

SEQ ID NO: 2 - DNA, 61 bp, Homo sapiens
tcaattttagaatgtcagggctgtctatggmatattccaaacctcgacattcaaagtggca

SEQ ID NO: 3 - DNA, 201 bp, Homo sapiens
cctaccaggcagaggtgtttaaccccttcgttggcgagccagctcctccaggacacagccatgccgccggccccgccgcgctgcgctctgtgcccgccggmccgagaaggagcgggcggcggccggggcagcggttacagttgtgcggcctgccgggccgctgagcgaggaccgagggtcaaagactgagtggaagcccga

SEQ ID NO: 4 - DNA, 201 bp, Homo sapiens
cgtcctgtgggtcttgagcagcagacagtttctttctgcctggacccccgcccccaccccaaaagaggccacagagcttcagcaggaagtttggcctcccygcccgtctccagggaagcagcttttggtccccatctggggcaagcctccatgcccaaacatggtcaagtctgagcacacagcctggagacacagactcgg

SEQ ID NO: 5 - DNA, 201 bp, Homo sapiens
ggccttaccttcgcgcctgcagccgcaggtcctgctctgaggggctgaacacatgctggagctggtgcttggcaattgcctgccacttgcctctgttttcwcgctccagccgctcccagatttctgggatctaggagagagaagtggagagtggcaggaaggtgctggtaaagtgggacagtggtcctgagcagctaactt

SEQ ID NO: 6 - DNA, 201 bp, Homo sapiens
gaaaaccctccagtcagcgcttatcccttctgctctctcccctcacccagagaaatacatggagtttgaccttaatggaaatggcgatattggtgagaaaygggtgatttgcgggggcagggtggtgtgcaggcctaagaagacagaggtctctcctacatgctccattcctcatgatttgggagggggcccacctaccac

SEQ ID NO: 7 - DNA, 201 bp, Homo sapiens
aaaattaggagtggaattactgagtcatagggtatgcatgtgttcaacttttgtggatgtttccacatgattttccaaagtgaaaccctgttttgagatckggaatattgactgtctttcactctccttggaccctagggctacagaactggctacccttatcgataccctgctctgagagagaagtctaaaaaatacagc

SEQ ID NO: 8 - DNA, 201 bp, Homo sapiens
gctctaattcatgcaattaacgccttctgtatgaaacagtttttcctcctttctttaagggggtggggatacaaaagtaactcagaaaattttctttgtcmtaaaactacactgaacatgtgaatagcatattgtggtggacaagagcaagagtaaacagatgaaaagaataaatgtttagatttgttgataaaacaggaa

(19) United States
(12) Patent Application Publication
(10) Pub. No.: US 2007/0065865 A1
(43) Pub. Date: Mar. 22, 2007

(54) Polymorphisms Associated with Coronary Artery Disease

(75) Inventors: Victoria E.H. Carlton, San Francisco, CA (US); Martin Emilio Moorhead, San Mateo, CA (US); Chunnuan Chen, Sunnyvale, CA (US); Malek Faham, Pacifica, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/530,407

(22) Filed: Sep. 8, 2006

Related U.S. Application Data
(60) Provisional application No. 60/715,038, filed on Sep. 8, 2005.

(51) Int. Cl. C12Q 1/68

(52) U.S. Cl. 435/6

(57) ABSTRACT

The invention provides human polymorphisms that are associated with coronary artery disease (CAD). Polymorphisms in genes were identified that confer an increased susceptibility to CAD or a decreased susceptibility. Particular alleles of the polymorphisms were identified as being associated with differential risk. In particular an allele of CDC42 in combination with an allele of PARD3 was shown to confer increased risk of CAD.

### RELATED APPLICATIONS

This application is related to U.S. Provisional Patent Application No. 60/715,038, filed Sep. 8, 2005, the entire disclosure of which is incorporated herein by reference in its entirety.

### FIELD OF THE INVENTION

The invention is related to polymorphisms associated with coronary artery disease.

### BACKGROUND OF THE INVENTION

Coronary artery disease (CAD) occurs when the arteries that supply blood to the heart muscle (coronary arteries) become hardened and narrowed. The arteries harden and become narrow due to the buildup of plaque on the inner walls or lining of the arteries (atherosclerosis). Blood flow to the heart is reduced as plaque narrows the coronary arteries. This decreases the oxygen supply to the heart muscle. CAD is the most common type of heart disease. It is the leading cause of death in the U.S. in both men and women. When blood flow and oxygen supply to the heart are reduced or cut off, you can develop anginia or heart attack. Angina is chest pain or discomfort that occurs when your heart is not getting enough blood. A heart attack happens when a blood clot suddenly cuts off most or all blood supply to part of the heart. Cells in the heart muscle that do not receive enough oxygen-carrying blood begin to die. This can cause permanent damage to the heart muscle. Over time, CAD can weaken your heart muscle and contribute to heart failure and arrhythmias. In heart failure, the heart is not able to pump blood to the rest of the body effectively. Heart failure does not mean that your heart has stopped or is about to stop working. But it does mean that your heart is failing to pump blood the way that it should. Arrhythmias are changes in the normal rhythm of the heartbeats. Some can be quite serious. (National Institutes of Health website, 2003)

Despite the clear role of lifestyle in CAD, family history has also long been recognized as a risk factor, particularly in disease with onset before age 70 (Hunt, S. C., R. R. Williams, and G. K. Barlow, A comparison of positive family history definitions for defining risk of future disease. J Chronic Dis, 1986. 39(10): 809-821). Heritability of the trait is estimated to be ˜0.34 (Williams, F. M., et al., A common genetic factor underlies hypertension and other cardiovascular disorders. BMC Cardiovasc Disord, 2004. 4(1): 20).

### SUMMARY OF THE INVENTION

The invention provides methods and compositions for determining susceptibility for coronary artery disease, or related conditions, in an individual. In one aspect, the invention provides nucleic acid sequences that may be used to determine the presence or absence of nucleotides at polymorphic sites in an individual's RNA or genomic DNA that are associated with susceptibility for coronary artery disease, or related conditions. In another aspect, the invention provides a method and kits for identifying a patient having a susceptibility to coronary artery disease having the following steps: (i) obtaining a sample of DNA or RNA from a patient; and (ii) detecting in the sample one or more at-risk polymorphisms located in a sequence of one or more genes selected from the group consisting of those listed in Table 1. In one aspect, the one or more genes are selected from the group consisting of PARD3 and CDC42 (ENSG0000007083 1).

As used herein, an “at-risk polymorphism” is a polymorphism having an association with the presence of coronary artery disease in an individual. In another aspect, an “at-risk polymorphism” is a polymorphism in linkage disequilibrium with a polymorphism listed in Table 1. In still another aspect, an “at-risk polymorphism” is a polymorphism in PARD3 or CDC42. In still another aspect, an “at-risk polymorphism” is a polymorphism in PARD3 or CDC42 listed in Table 1. In preferred aspects one allele of a biallelic polymorphism is identified as being associated with either an increased risk of CAD, called herein an “at-risk allele” or a decreased risk of CAD (conferring a protective effect). The “at-risk allele” may be the major or the minor allele.

In one aspect, SNPs identified in ABCA1 that showed significant allelic associated with CAD were re3758294 and rs4149265. The minor alleles of both appear to be protective and are in high LD with each other.

In another aspect of the invention, it has been discovered that CDC42 and PARD3 act together to increase risk of CAD. Individuals who carry at-risk polymorphisms at both rs16826506 (CDC42) and rs1545214 (PARD3) are at about six times the risk of developing CAD than someone who has wild-type alleles at both loci, as determined by a 2-sided Fisher Exact Test p-value of 0.006. The rare minor allele of rs16826506 is far more common in cases than controls

### DEFINITIONS

Terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

“Amplicon” means the product of a polynucleotide amplification reaction. That is, it is a population of polynucleotides, usually double stranded, that are replicated from one or more starting sequences. The one or more starting sequences may be one or more copies of the same sequence, or it may be a mixture of different sequences. Amplicons may be produced by a variety of amplification reactions whose products are multiple replicates of one or more target nucleic acids. Generally, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references that are incorporated herein by reference: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like.

“Complementary or substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

“Coronary artery disease” refers to a disease of the heart and the coronary arteries that is characterized by atherosclerotic arterial deposits that block blood flow to the heart.

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. In one aspect, stable duplex means that a duplex structure is not destroyed by a stringent wash, e.g. conditions including tempature of about 5° C. less that the Tₘ of a strand of the duplex and low monovalent salt concentration, e.g. less than 0.2 M, or less than 0.1 M. “Perfectly matched” in reference to a duplex means that the poly- or oligonucleotide strands making up the duplex form a double stranded structure with one another such that every nucleotide in each strand undergoes Watson-Crick basepairing with a nucleotide in the other strand. The term “duplex” comprehends the pairing of nucleoside analogs, such as deoxyinosine, nucleosides with 2-aminopurine bases, PNAs, and the like, that may be employed. A “mismatch” in a duplex between two oligonucleotides or polynucleotides means that a pair of nucleotides in the duplex fails to undergo Watson-Crick bonding.

“Genetic locus,” or “locus” in reference to a genome or target polynucleotide, means a contiguous subregion or segment of the genome or target polynucleotide. As used herein, genetic locus, or locus, may refer to the position of a nucleotide, a gene, or a portion of a gene in a genome, including mitochondrial DNA, or it may refer to any contiguous portion of genomic sequence whether or not it is within, or associated with, a gene. In one aspect, a genetic locus refers to any portion of genomic sequence, including mitochondrial DNA, from a single nucleotide to a segment of few hundred nucleotides, e.g. 100-300, in length. Usually, a particular genetic locus may be identified by its nucleotide sequence, or the nucleotide sequence, or sequences, of one or both adjacent or flanking regions.

“Hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1M, more usually less than about 500 mM and less than about 200 mM. Hybridization temperatures can be as low as 50° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tₘ for the specific sequence at s defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2^{nd} Ed. Cold Spring Harbor Press (1989) and Anderson “Nucleic Acid Hybridization” 1^{st} Ed., BIOS Scientific Publishers Limited (1999), which are hereby incorporated by reference in its entirety for all purposes above. “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

“Hybridization-based assay” means any assay that relies on the formation of a stable duplex or triplex between a probe and a target nucleotide sequence for detecting or measuring such a sequence. In one aspect, probes of such assays anneal to (or form duplexes with) regions of target sequences in the range of from 8 to 100 nucleotides; or in other aspects, they anneal to target sequences in the range of from 8 to 40 nucleotides, or more usually, in the range of from 8 to 20 nucleotides. A “probe” in reference to a hybridization-based assay mean a polynucleotide that has a sequence that is capable of forming a stable hybrid (or triplex) with its complement in a target nucleic acid and that is capable of being detected, either directly or indirectly. Hybridization-based assays include, without limitation, assays based on use of oligonucleotides, such as polymerase chain reactions, NASBA reactions, oligonucleotide ligation reactions, single-base extensions of primers, circularizable probe reactions, allele-specific oligonucleotides hybridizations, either in solution phase or bound to solid phase supports, such as microarrays or microbeads. There is extensive guidance in the literature on hybridization-based assays, e.g. Hames et al, editors, Nucleic Acid Hybridization a Practical Approach (IRL Press, Oxford, 1985); Tijssen, Hybridization with Nucleic Acid Probes, Parts I & II (Elsevier Publishing Company, 1993); Hardiman, Microarray Methods and Applications (DNA Press, 2003); Schena, editor, DNA Microarrays a Practical Approach (IRL Press, Oxford, 1999); and the like. In one aspect, hybridization-based assays are solution phase assays; that is, both probes and target sequences hybridize under conditions that are substantially free of surface effects or influences on reaction rate. A solution phase assay may include circumstance where either probes or target sequences are attached to microbeads.

“Linkage” describes the tendency of genes, alleles, loci or genetic markers to be inherited together as a result of their location on the same chromosome, and can be measured by percent recombination between the genes, alleles, loci or genetic markers that are physically-linked on the same chromosome. Loci occurring within 50 centimorgan of each other are linked. Some linked markers occur within the same gene or gene cluster.

“Kit” refers to any delivery system for delivering materials or reagents for carrying out a method of the invention. In the context of assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials for assays of the invention. In one aspect, kits of the invention comprise probes specific for interfering polymorphic loci. In another aspect, kits comprise nucleic acid standards for validating the performance of probes specific for interfering polymorphic loci. Such contents may be delivered to the intended recipient together or separately. For example, a first container may contain an enzyme for use in an assay, while a second container contains probes.

“Ligation” means to form a covalent bond or linkage between the termini of two or more nucleic acids, e.g. oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon of a terminal nucleotide of one oligonucleotide with 3′ carbon of another oligonucleotide. A variety of template-driven ligation reactions are described in the following references, which are incorporated by reference: Whitely et al, U.S. Pat. No. 4,883,750; Letsinger et al, U.S. Pat. No. 5,476,930; Fung et al, U.S. Pat. No. 5,593,826; Kool, U.S. Pat. No. 5,426,180; Landegren et al, U.S. Pat. No. 5,871,921; Xu and Kool, Nucleic Acids Research, 27: 875-881 (1999); Higgins et al, Methods in Enzymology, 68: 50-71 (1979); Engler et al, The Enzymes, 15: 3-29 (1982); and Namsaraev, U.S. patent publication 2004/0110213.

“Microarray” refers to a solid phase support having a planar surface, which carries an array of nucleic acids, each member of the array comprising identical copies of an oligonucleotide or polynucleotide immobilized to a spatially defined region or site, which does not overlap with those of other members of the array; that is, the regions or sites are spatially discrete. Spatially defined hybridization sites may additionally be “addressable” in that its location and the identity of its immobilized oligonucleotide are known or predetermined, for example, prior to its use. Typically, the oligonucleotides or polynucleotides are single stranded and are covalently attached to the solid phase support, usually by a 5′-end or a 3′-end. The density of non-overlapping regions containing nucleic acids in a microarray is typically greater than 100 per cm², and more preferably, greater than 1000 per cm². Microarray technology is reviewed in the following references: Schena, Editor, Microarrays: A Practical Approach (IRL Press, Oxford, 2000); Southern, Current Opin. Chem. Biol., 2: 404-410 (1998); Nature Genetics Supplement, 21: 1-60 (1999). As used herein, “random microarray” refers to a microarray whose spatially discrete regions of oligonucleotides or polynucleotides are not spatially addressed. That is, the identity of the attached oligonucleotides or polynucleotides is not discernable, at least initially, from its location. In one aspect, random microarrays are planar arrays of microbeads wherein each microbead has attached a single kind of hybridization tag complement, such as from a minimally cross-hybridizing set of oligonucleotides. Arrays of microbeads may be formed in a variety of ways, e.g. Brenner et al, Nature Biotechnology, 18: 630-634 (2000); Tulley et al, U.S. Pat. No. 6,133,043; Stuelpnagel et al, U.S. Pat. No. 6,396,995; Chee et al, U.S. Pat. No. 6,544,732; and the like. Likewise, after formation, microbeads, or oligonucleotides thereof, in a random array may be identified in a variety of ways, including by optical labels, e.g. fluorescent dye ratios or quantum dots, shape, sequence analysis, or the like.

“Nucleoside” as used herein includes the natural nucleosides, including 2′-deoxy and 2′-hydroxyl forms, e.g. as described in Kornberg and Baker, DNA Replication, 2nd Ed. (Freeman, San Francisco, 1992). “Analogs” in reference to nucleosides includes synthetic nucleosides having modified base moieties and/or modified sugar moieties, e.g. described by Scheit, Nucleotide Analogs (John Wiley, New York, 1980); Uhlman and Peyman, Chemical Reviews, 90: 543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al, Exp. Opin. Ther. Patents, 6: 855-870 (1996); Mesmaeker et al, Current Opinion in Structual Biology, 5: 343-355 (1995); and the like. Exemplary types of polynucleotides that are capable of enhancing duplex stability include oligonucleotide N3′→P5′ phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (LNAs), and like compounds. Such oligonucleotides are either available commercially or may be synthesized using methods described in the literature.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999)(two-color real-time PCR). Usually, distinct sets of primers are employed for each sequence being amplified. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references that are incorporated by reference: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); Becker-Andre et al, Nucleic Acids Research, 17: 9437-9446 (1989); and the like.

“Polymorphism” refers to the occurrence of two or more genetically determined alternative sequences or alleles in a population. A polymorphic marker or site is the locus at which such polymorphism occurs. Preferred markers have at least two alleles, each occurring at frequency of greater than 1%, and more preferably greater than 5% or 10% of a selected population. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, deletions, simple sequence repeats, and insertion elements, such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms.

“Polymorphism” or “genetic variant” means a substitution, inversion, insertion, or deletion of one or more nucleotides at a genetic locus, or a translocation of DNA from one genetic locus to another genetic locus. In one aspect, polymorphism means one of multiple alternative nucleotide sequences that may be present at a genetic locus of an individual and that may comprise a nucleotide substitution, insertion, or deletion with respect to other sequences at the same locus in the same individual, or other individuals within a population. An individual may be homozygous or heterozygous at a genetic locus; that is, an individual may have the same nucleotide sequence in both alleles, or have a different nucleotide sequence in each allele, respectively. In one aspect, insertions or deletions at a genetic locus comprises the addition or the absence of from 1 to 10 nucleotides at such locus, in comparison with the same locus in another individual of a population (or another allele in the same individual). Usually, insertions or deletions are with respect to a major allele at a locus within a population, e.g. an allele present in a population at a frequency of fifty percent or greater.

“Single nucleotide polymorphism” or “SNP” occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

“Isolated nucleic acid” means an object species invention that is the predominant species present (i.e., on a molar basis it is more abundant than any other individual species in the composition). Preferably, an isolated nucleic acid comprises at least about 50 percent (on a molar basis) of all macromolecular species present; and more preferably, an isolated nucleic acid comprises at least about 90 percent (on a molar basis) of all macromolecular species present. Most preferably, the object species is purified to essential homogeneity (contaminant species cannot be detected in the composition by conventional detection methods).

“Linkage disequilibrium” or “LD” or “allelic association” or “association” means the preferential association of a particular allele or genetic marker with a specific allele, or genetic marker at another chromosomal location more frequently than expected by chance given the particular allele frequency in the population. For example, if locus X has alleles a and b, which occur equally frequently, and another locus Y has alleles c and d, which occur equally frequently, one would expect the haplotype ac to occur with a frequency of 0.25 in a population of individuals. If ac occurs more frequently, then alleles a and c are considered in linkage disequilibrium. Linkage disequilibrium may result from natural selection of certain combination of alleles, through the admixture of two or more genetically different populations or because an allele has been introduced into a population too recently to have reached equilibrium (random association) between linked alleles.

A “marker” or “biomarker” in linkage disequilibrium with disease predisposing variants can be particularly useful in detecting susceptibility to disease (or association with sub-clinical phenotypes) notwithstanding that the marker does not cause the disease. For example, a marker (X) that is not itself a causative element of a disease, but which is in linkage disequilibrium with a gene (including regulatory sequences) (Y) that is a causative element of a phenotype, can be used detected or indicate susceptibility to the disease in circumstances in which the gene Y may not have been identified or may not be readily detectable. Younger alleles (i.e., those arising from mutation relatively recently) are expected to have a larger genomic segment in linkage disequilibrium. The age of an allele can be determined from whether the allele is shared among different human ethnic groups and/or between humans and related species.

A “study population” can consist of any number of individuals, subjects or biological samples that may come from human or non-human organisms.

“Polynucleotide” or “oligonucleotide” are used interchangeably and each mean a linear polymer of nucleotide monomers. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidie linkages may be naturally occurring or may be analogs thereof, e.g. naturally occurring or non-naturally occurring analogs. Non-naturally occurring analogs may include PNAs, phosphorothioate internucleosidie linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moities, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references.

“Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of from 14 to 36 nucleotides.

“Readout” means a parameter, or parameters, which are measured and/or detected that can be converted to a number or value. In some contexts, readout may refer to an actual numerical representation of such collected or recorded data. For example, a readout of fluorescent intensity signals from a microarray is the address and fluorescence intensity of a signal being generated at each hybridization site of the microarray; thus, such a readout may be registered or stored in various ways, for example, as an image of the microarray, as a table of numbers, or the like.

“Solid support”, “support”, and “solid phase support” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. Microarrays usually comprise at least one planar solid phase support, such as a glass microscope slide.

“Specific” or “specificity” in reference to the binding of one molecule to another molecule, such as a labeled target sequence for a probe, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecule in a reaction or sample, it forms the largest number of the complexes with the second molecule. Preferably, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak non-covalent chemical interactions, such as Van der Waal forces, hydrogen bonding, base-stacking interactions, ionic and hydrophobic interactions, and the like, dominate the interaction of the molecules.

“Tₘ” is used in reference to “melting temperature.” Melting temperature is the temperature at which a population of double-stranded nucleic acid molecules becomes half dissociated into single strands. Several equations for calculating the Tm of nucleic acids are well known in the art. As indicated by standard references, a simple estimate of the Tm value may be calculated by the equation. Tm=81.5+0.41 (% G+C), when a nucleic acid is in aqueous solution at 1 M NaCl (see e.g., Anderson and Young, Quantitative Filter Hybridization, in Nucleic Acid Hybridization (1985). Other references (e.g., Allawi, H. T. & SantaLucia, J., Jr., Biochemistry 36, 10581-94 (1997)) include alternative methods of computation which take structural and environmental, as well as sequence characteristics into account for the calculation of Tm.

“Sample” means a quantity of material from a biological, environmental, medical, or patient source in which detection or measurement of target nucleic acids is sought. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may include materials taken from a patient including, but not limited to cultures, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

### DETAILED DESCRIPTION OF THE INVENTION

The invention provides a collection of novel polymorphisms in genes encoding products related to susceptibility and/or course or outcome of coronary artery disease. Detection of polymorphisms in such genes is useful in designing and performing diagnostic assays for evaluation of genetic risks for coronary artery disease and other related conditions. Analysis of such polymorphisms is also useful in designing prophylactic and therapeutic regimes customized to underlying abnormalities. Detection of such polymorphisms is also useful for conducting clinical trials of drugs for treatment of these diseases and the underlying biological abnormalities. The polymorphisms of the invention also have more general applications, such as forensics, paternity testing, linkage analysis and positional cloning.

### Polymorphisms of the Invention

A study was designed to assess common alleles in 237 candidate genes for association with CAD and to assess rare alleles in a subset of such candidate genes for association with CAD. Common alleles were measured using molecular inversion probes, which are disclosed in the following references that are incorporated by reference: Willis et al, U.S. Pat. No. 6,858,412; Hardenbol et al, Nature Biotechnology, 21: 673-678 (2003); and Hardenbol et al, Genome Research, 15: 269-275 (2005). Rare alleles were identified using mismatch repair detection, a polymorphism discovery technique disclosed in the following references that are incorporated by reference: Faham et al, U.S. patent publication 2003/0003472; Faham et al, Human Molecular Genetics, 10: 1657-1664 (2001); and Fakhrair-Rad et al, Genome Research, 14: 1404-1412 (2004). After discovery, rare single nucleotide polymorphisms were measured using molecular inversion probe technology.

Analyzed samples included DNA obtained from 314 cases and 314 controls. The enrollment criteria for cases were as follows: US White Caucasians; CAD with 70% occlusion or greater in at least one artery. Cases were excluded if any of the following applied: diagnosed with type 2 diabetes; MI (heart attack); extreme hypertension (systolic bp>180 or diastolic bp>100 or with end-organ damage); hypotension (systolic bp<90 and diastolic bp<50); heavy smokers (current users of greater than one pack per day). Controls were drawn from a group of healthy US White Caucasians. Exclusion criteria were as follows: first degree-relatives with type 2 diabetes; hypertension (systolic bp>140 or diastolic bp>90); fasting glucose>126 mg/dl; total cholesterol=>300 mg/dl. Controls were matched to cases with the following criteria: exact match for gender; age match control is −3/+6 years of the case; body mass index (BMI) match is ±3 units.

Measures of the association of polymorphisms in cases with CAD were obtained by applying chi-squared tests and Fisher's exact tests to the data as follows: (i) p-value of chi-square of 2-by-2 allele count table (designated “P-Value Allele Chiˆ2”), (ii) Fisher Exact test p-value (2-sided) of 2-by-2 allele count table (designated “P-Value Allele Fisher”), (iii) p-value of chi-square of 3-by-2 genotype count table (designated “P-Value Genotype Chiˆ2”), (iv) Fisher Exact test p-value (2-sided) of 2-by-2 genotype count table assuming recessive model (minor/minor vs all others)(designated “P-Value Recessive Fisher”), and (v) Fisher Exact test p-value (2-sided) of 2-by-2 genotype count table assuming dominant model (carriers of minor allele vs all others) (designated “P-Value Dominant Fisher”). If any of the resulting measures was equal to or less than 0.05, the polymorphism was deemed to have a significant association with CAD. Such polymorphisms are listed in Table 1. Column designations (in addition to those described above) are as follows: “SNP” is a reference SNP (or “refSNP”) cluster identifier or an MRD ID (which are further identified by sequence in Table 2); “Gene” is a HUGO or Ensembl ID (as of May 2005); “Chrom” is chromosome number; “Posn” is the base position on the indicated chromosome (NCBI build 35); “Major Allele” is self-explanatory; “Minor Allele” is self-explanatory; “1 Case” is the count of major allele in cases; “2 Case” is the count of minor allele in cases; “1 Cntr” is the count of major allele in controls; “2 Cntr” is the count of minor allele in controls; “1/1 Case” is the count of major/major genotype in cases; “1/2 Case” is the count of major/minor genotype in cases; “2/2 Case” is the count of minor/minor genotype in cases; “1/1 Cntr” is the count of major/major genotype in controls; “1/2 Cntr” is the count of major/minor genotype in controls; and “2/2 Cntr” is the count of minor/major genotype in controls. SNPs in Table 2 were identified in the study. The SNP alleles are as follows: M is A or C, Y is C or T, W is A or T and K is G or T.

In one aspect, SNPs in CDC42 and PARD3 were found to act together to increase risk of CAD. Individuals who carry at-risk polymorphisms at both rs16826506 (CDC42) and rs1545214 (PARD3) are at about six times the risk of developing CAD than someone who has wild-type alleles at both loci, as determined by a 2-sided Fisher Exact Test p-value of 0.006. SNP rs16826506 is found in the following sequence: SEQ ID NO 1: 5′-aagtaatggt atattaaatt tggaatatag Mgaaaacaat gacccataat gtcatgataa a-3′ where the major allele is A and the minor allele is C. SNP rs1545214 is found in the following sequence SEQ ID NO 2: 5′-tcaattttag aatgtcaggg ctgtctatgg Matattccaa acctcgacat tcaaagtggc a-3′ with major allele C and minor allele A. In some aspects the patient may be identified at being at increased risk for CAD if the patient is heterozygous at both SNPs rs1545214 and rs16826506, but the patient may also be identified as being at increased risk if the patient is homozygous for the minor allele of one or both SNPs.

### Analysis of Polymorphisms

A. Preparation of Samples. Polymorphisms are detected in a target nucleic acid from an individual being analyzed. For assay of genomic DNA, virtually any biological sample (other than pure red blood cells) is suitable. For example, convenient tissue samples include whole blood, semen, saliva, tears, urine, fecal material, sweat, buccal epithelium, skin and hair. For assay of cDNA or mRNA, the tissue sample must be obtained from an organ in which the target nucleic acid is expressed.

Many of the methods described below require amplification of DNA from target samples. This can be accomplished by e.g., PCR. See generally PCR Technology: Principles and Applications for DNA Amplification (ed. H. A. Erlich, Freeman Press, NY, NY, 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202 (each of which is incorporated by reference for all purposes).

Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

B. Single Base Extension Methods for Detecting Polymorphisms. Single base extension methods are described by e.g., U.S. Pat. No. 5,846,710, U.S. Pat. No. 6,004,744, U.S. Pat. No. 5,888,819 and U.S. Pat. No. 5,856,092. In brief, the methods work by hybridizing a primer that is complementary to a target sequence such that the 3′ end of the primer is immediately adjacent to but does not span a site of potential variation in the target sequence. That is, the primer comprises a subsequence from the complement of a target polynucleotide terminating at the base that is immediately adjacent and 5′ to the polymorphic site. The hybridization is performed in the presence of one or more labeled nucleotides complementary to base(s)that may occupy the site of potential variation. For example, for a biallelic polymorphisms two differentially labeled nucleotides can be used. For a tetraallelic polymorphism four differentially labeled nucleotides can be used. In some methods, particularly methods employing multiple differentially labeled nucleotides, the nucleotides are dideoxynucleotides. Hybridization is performed under conditions permitting primer extension if a nucleotide complementary to a base occupying the site of variation in the target sequence is present. Extension incorporates a labeled nucleotide thereby generating a labeled extended primer. If multiple differentially labeled nucleotides are used and the target is heterozygous then multiple differentially labeled extended primers can be obtained. Extended primers are detected providing an indication of which bas(es) occupy the site of variation in the target polynucleotide.

C. Allele-Specific Probes. The design and use of allele-specific probes for analyzing polymorphisms is described by e.g., Saiki et al., Nature 324, 163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15 mer at the 7 position; in a 16 mer, at either the 8 or 9 position) of the probe. This design of probe achieves good discrimination in hybridization between different allelic forms. Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence. The polymorphisms can also be identified by hybridization to nucleic acid arrays, some example of which are described by WO 95/11995 (incorporated by reference in its entirety for all purposes).

D. Allele-Specific Amplification Methods. An allele-specific primer hybridizes to a site on target DNA overlapping a polymorphism and only primes amplification of an allelic form to which the primer exhibits perfect complementarily. See Gibbs, Nucleic Acid Res. 17, 2427-2448 (1989). This primer is used in conjunction with a second primer that hybridizes at a distal site. Amplification proceeds from the two primers leading to a detectable product signifying the particular allelic form is present. A control is usually performed with a second pair of primers, one of which shows a single base mismatch at the polymorphic site and the other of which exhibits perfect complementarily to a distal site. The single-base mismatch prevents amplification and no detectable product is formed. In some methods, the mismatch is included in the 3′-most position of the oligonucleotide aligned with the polymorphism because this position is most destabilizing to elongation from the primer. See, for example, WO 93/22456.

E. Direct-Sequencing. The direct analysis of the sequence of polymorphisms of the present invention can be accomplished using either the dideoxy-chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

F. Denaturing Gradient Gel Electrophoresis. Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W. H. Freeman and Co, New York, 1992), Chapter 7.

G. Single-Strand Conformation Polymorphism Analysis. Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures that are partially dependent on the base sequence. The different electrophoretic mobilities of single-stranded amplification products can be related to base-sequence difference between alleles of target sequences.

### Methods of Use

After determining polymorphic form(s) present in an individual at one or more polymorphic sites, this information can be used in a number of methods.

A. Association Studies with CAD

The polymorphisms of the invention may contribute to the phenotype of an organism in different ways. Some polymorphisms occur within a protein coding sequence and contribute to phenotype by affecting protein structure. The effect may be neutral, beneficial or detrimental, or both beneficial and detrimental, depending on the circumstances. By analogy, a heterozygous sickle cell mutation confers resistance to malaria, but a homozygous sickle cell mutation causes severe disease. Other polymorphisms occur in non-coding regions but may exert phenotypic effects indirectly via influence on replication, transcription, and translation. A single polymorphism may affect more than one phenotypic trait. Likewise, a single phenotypic trait may be affected by polymorphisms in different genes.

Correlation is performed for a population of individuals who have been tested for the presence or absence of CAD or an intermediate phenotype and for one or more polymorphic markers or polymorphic sites. To perform such analysis, the presence or absence of a set of polymorphic forms (i.e. a polymorphic set) is determined for a set of the individuals, some of whom exhibit a particular trait, and some of whom may exhibit lack of the trait. The alleles of each polymorphism of the set are then reviewed to determine whether the presence or absence of a particular allele is associated with the trait of interest. Correlation can be performed by standard statistical methods including, but not limited to, chi-squared test, Fisher Exact Test, Analysis of Variance, contingency table tests, logistic regression, parametric linkage analysis, non-parametric linkage analysis etc and statistically significant correlations between polymorphic form(s) and phenotypic characteristics are noted. For example, it might be found that the presence of allele A1 at polymorphism A correlates with CAD, measured either as a categorical or continuous trait. As a further example, it might be found that the combined presence of allele A1 at polymorphism A and allele B1 at polymorphism B correlates with CAD or a sub-phenotype.

B. Diagnosis of CAD

Polymorphic forms that correlate with CAD or sub-phenotypes are useful in diagnosing CAD or susceptibility thereto or predicting disease prognosis. Combined detection of several such polymorphic forms typically increases the probability of an accurate diagnosis. For example, the presence of a single polymorphic form known to correlate with CAD might indicate a probability of 20% that an individual has or is susceptible to CAD, whereas detection of five polymorphic forms, each of which correlates with less than 20% probability, might indicate a probability up to 80% that an individual has or is susceptible to CAD. Analysis of the polymorphisms of the invention can be combined with that of other polymorphisms or other risk factors for CAD, such as family history. Polymorphisms could be used to diagnose CAD or sub-phenotypes of CAD at the pre-symptomatic stage, as a method of post-symptomatic diagnosis, as a method of confirmation of diagnosis or as a post-mortem diagnosis.

Patients diagnosed with CAD can be treated with conventional therapies and/or can be counseled to avoid environmental factors that exacerbate the condition. Patients diagnosed with CAD may also be counseled about the risk of genetically transmitting the disease to offspring, or counseled about the risk of family members sharing genetic variation(s) relevant to CAD.

C. Drug Screening

The polymorphism(s) showing the strongest correlation with CAD within a given gene are likely either to have a causative role in the manifestation of the phenotype or to be in linkage disequilibrium with the causative variants. Such a role can be confirmed by in vitro gene expression of the variant gene or by producing a transgenic animal expressing a human gene bearing such a polymorphism and determining whether the animal develops the phenotype. Polymorphisms in coding regions that result in amino acid changes usually cause CAD by decreasing, increasing or otherwise altering the activity of the protein encoded by the gene in which the polymorphism occurs. Polymorphisms in coding regions that introduce stop codons usually cause CAD by reducing (heterozygote) or eliminating (homozygote) functional protein produced by the gene. Occasionally, stop codons result in production of a truncated peptide with aberrant activities relative to the full-length protein. Polymorphisms in regulatory regions typically cause CAD or related phenotypes by causing increased or decreased expression of the protein encoded by the gene in which the polymorphism occurs. Polymorphisms in exonic or untranslated sequences can cause CAD or related phenotypes either through the same mechanism as polymorphisms in regulatory sequences or by causing altered spliced patterns resulting in an altered protein.

Having identified certain polymorphisms as having causative roles in CAD, and having elucidated at least in general terms whether such polymorphisms increase or decrease the activity or expression level of associated proteins, customized therapies can be devised for classes of patients with different genetic subtypes of diseases. For example, if a polymorphism in a given protein causes CAD by increasing the expression level or activity of the protein, the diseases associated with the polymorphism can be treated by administering an antagonist of the protein. If a polymorphism in a given protein causes CAD by decreasing the expression level or activity of a protein, the form of CAD associated with the polymorphism can be treated by administering the protein itself, a nucleic acid encoding the protein that can be expressed in a patient, or an analog or agonist of the protein.

The polymorphisms of the invention are also useful for conducting clinical trials of drug candidates for CAD. Such trials are performed on treated or control populations having similar or identical polymorphic profiles at a defined collection of polymorphic sites. Use of genetically matched populations eliminates or reduces variation in treatment outcome due to genetic factors, leading to a more accurate assessment of the efficacy of a potential drug.

Furthermore, the polymorphisms of the invention may be used after the completion of a clinical trial to elucidate differences in response to a given treatment. For example, the set of polymorphisms may be used to stratify the enrolled patients into disease sub-types or classes. It may further be possible to use the polymorphisms to identify subsets of patients with similar polymorphic profiles who have unusual (high or low) response to treatment or who do not respond at all (non-responders). In this way, information about the underlying genetic factors influencing response to treatment can be used in many aspects of the development of treatment (these range from the identification of new targets, through the design of new trials to product labeling and patient targeting). Additionally, the polymorphisms may be used to identify the genetic factors involved in adverse response to treatment (also called adverse events or adverse drug reactions). For example, patients who show adverse response may have more similar polymorphic profiles than would be expected by chance. This would allow the early identification and exclusion of such individuals from treatment. It would also provide information that might be used to understand the biological causes of adverse events and to modify the treatment to avoid such outcomes.

Even if a polymorphism is not causative but is instead in linkage disequilibrium with a causative variant, it may still be useful for genetic tests, including diagnosis, prognosis, and drug screening.

D. Forensics

Determination of which polymorphic forms occupy a set of polymorphic sites in an individual identifies a set of polymorphic forms that distinguishes the individual. See generally National Research Council, The Evaluation of Forensic DNA Evidence (Eds. Pollard et al., National Academy Press, DC, 1996). The more sites that are analyzed the lower the probability that the set of polymorphic forms in one individual is the same as that in an unrelated individual. Preferably, if multiple sites are analyzed, the sites are unlinked. Thus, polymorphisms of the invention are often used in conjunction with polymorphisms in distal genes. Preferred polymorphisms for use in forensics are diallelic because the population frequencies of two polymorphic forms can usually be determined with greater accuracy than those of multiple polymorphic forms at multi-allelic loci.

The capacity to identify a distinguishing or unique set of forensic markers in an individual is useful for forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental error) that the suspect was not the source of the sample. If the set of markers does match, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance.

p(ID) is the probability that two random individuals have the same polymorphic or allelic form at a given polymorphic site. In diallelic loci, four genotypes are possible: AA, AB, BA, and BB. If alleles A and B occur in a haploid genome of the organism with frequencies x and y, the probability of each genotype in a diploid organism are (see WO 95/12607):
Homozygote: p(AA)=x²
Homozygote: p(BB)=y²=(1−x)²
Single Heterozygote: p(AB)=p(BA)=xy=x(1−x)
Both Heterozygotes: p(AB+BA)=2xy=2x(1−x)

The probability of identity at one locus (i.e., the probability that two individuals, picked at random from a population will have identical polymorphic forms at a given locus) is given by the equation:
p(ID)=(x²)²+(2xy)²+(y²)².

These calculations can be extended for any number of polymorphic forms at a given locus. For example, the probability of identity p(ID) for a 3-allele system where the alleles have the frequencies in the population of x, y and z, respectively, is equal to the sum of the squares of the genotype frequencies:
p(ID)=x⁴+(2xy)²+(2yz)²+(2xz)²+z⁴+y⁴

In a locus of n alleles, the appropriate binomial expansion is used to calculate p(ID) and p(exc).

The cumulative probability of identity (cum p(ID)) for each of multiple unlinked loci is determined by multiplying the probabilities provided by each locus.
cum p(ID)=p(ID1)p(ID2)p(ID3) . . . p(IDn)

The cumulative probability of non-identity for n loci (i.e. the probability that two random individuals will be different at 1 or more loci) is given by the equation:
cum p(nonID)=1−cum p(ID).

If several polymorphic loci are tested, the cumulative probability of non-identity for random individuals becomes very high (e.g., one billion to one). Such probabilities can be taken into account together with other evidence in determining the guilt or innocence of the suspect.

F. Paternity Testing

The object of paternity testing is usually to determine whether a male is the father of a child. In most cases, the mother of the child is known and thus, the mother's contribution to the child's genotype can be traced. Paternity testing investigates whether the part of the child's genotype not attributable to the mother is consistent with that of the putative father. Paternity testing can be performed by analyzing sets of polymorphisms in the putative father and the child.

If the set of polymorphisms in the child attributable to the father does not match the putative father, it can be concluded, barring experimental error, that the putative father is not the real biological father. If the set of polymorphisms in the child attributable to the father does match the set of polymorphisms of the putative father, a statistical calculation can be performed to determine the probability of coincidental match.

The probability of parentage exclusion (representing the probability that a random male will have a polymorphic form at a given polymorphic site that makes him incompatible as the father) is given by the equation (see WO 95/12607):
p(exc)=xy(1−xy)

where x and y are the population frequencies of alleles A and B of a diallelic polymorphic site.

(At a triallelic site p(exc)=xy(1−xy)+yz(1−yz)+xz(l-xz)+3xyz(1−xyz))), where x, y and z and the respective population frequencies of alleles A, B and C).

The probability of non-exclusion is
p(non-exc)=1−p(exc)

The cumulative probability of non-exclusion (representing the value obtained when n loci are used) is thus:
cum p(non-exc)=p(non-exc1)p(non-exc2)p(non-exc3) . . . p(non-excn)

The cumulative probability of exclusion for n loci (representing the probability that a random male will be excluded)
cum p(exc)=1−cum p(non-exc).

If several polymorphic loci are included in the analysis, the cumulative probability of exclusion of a random male is very high. This probability can be taken into account in assessing the liability of a putative father whose polymorphic marker set matches the child's polymorphic marker set attributable to his/her father.

G. Genetic Mapping of Phenotypic Traits

The polymorphisms shown in Table 2 can also be used to establish physical linkage between a genetic locus associated with a trait of interest and polymorphic markers that are not associated with the trait, but are in physical proximity with the genetic locus responsible for the trait and co-segregate with it. Such analysis is useful for mapping a genetic locus associated with a phenotypic trait to a chromosomal position, and thereby cloning gene(s) responsible for the trait. See Lander et al., Proc. Natl. Acad. Sci. (USA) 83, 7353-7357 (1986); Lander et al., Proc. Natl. Acad. Sci. (USA) 84, 2363-2367 (1987); Donis-Keller et al., Cell 51, 319-337 (1987); Lander et al., Genetics 121, 185-199 (1989)). Genes localized by linkage can be cloned by a process known as directional cloning. See Wainwright, Med. J. Australia 159, 170-174 (1993); Collins, Nature Genetics 1, 3-6 (1992) (each of which is incorporated by reference in its entirety for all purposes).

Linkage studies are typically performed on members of a family. Available members of the family are characterized for the presence or absence of a phenotypic trait and for a set of polymorphic markers. The distribution of polymorphic markers in an informative meiosis is then analyzed to determine which polymorphic markers co-segregate with a phenotypic trait. See, e.g., Kerem et al., Science 245, 1073-1080 (1989); Monaco et al., Nature 316, 842 (1985); Yamoka et al., Neurology 40, 222-226 (1990); Rossiter et al., FASEB Journal 5, 21-27 (1991).

Linkage is analyzed by calculation of LOD (log of the odds) values. A lod value is the relative likelihood of obtaining observed segregation data for a marker and a genetic locus when the two are located at a recombination fraction θ, versus the situation in which the two are not linked, and thus segregating independently (Thompson & Thompson, Genetics in Medicine (5th ed, W.B. Saunders Company, Philadelphia, 1991); Strachan, “Mapping the human genome” in The Human Genome (BIOS Scientific Publishers Ltd, Oxford), Chapter 4). A series of likelihood ratios are calculated at various recombination fractions (θ), ranging from θ=0.0 (coincident loci) to θ=0.50 (unlinked). Thus, the likelihood at a given value of θ is proportional to the probability of data if loci linked at θ to probability of data if loci unlinked. The computed likelihoods are usually expressed as the log (base10) of this ratio (i.e., a lod score). For example, a lod score of 3 indicates 1000:1 odds against an apparent observed linkage being a coincidence. The use of logarithms allows data collected from different families to be combined by simple addition. Computer programs are available for the calculation of lod scores for differing values of θ (e.g., LIPED, MLINK (Lathrop, Proc. Nat. Acad. Sci. (USA) 81, 3443-3446 (1984)). For any particular lod score, a recombination fraction may be determined from mathematical tables. See Smith et al., Mathematical tables for research workers in human genetics (Churchill, London, 1961); Smith, Ann. Hum. Genet. 32, 127-150 (1968). The value of θ at which the lod score is the highest is considered to be the best estimate of the recombination fraction. Positive lod score values suggest that the two loci are linked, whereas negative values suggest that linkage is less likely (at that value of θ) than the possibility that the two loci are unlinked. By convention, a combined lod score of +3 or greater (equivalent to greater than 1000:1 odds in favor of linkage) is considered definitive evidence that two loci are linked. Similarly, by convention, a negative lod score of −2 or less is taken as definitive evidence against linkage of the two loci being compared. Negative linkage data are useful in excluding a chromosome or a segment thereof from consideration. The search focuses on the remaining non-excluded chromosomal locations.

V. Kits

The invention further provides kits comprising assay components to implement detection assays specific for polymorphisms of the invention. Thus, for hybridization-based assays, such components include probes capable of forming stable duplexes with predetermined regions adjacent to or encompassing polymorphisms of the inventions. For assays that depend on a polymerase-based primer extension reaction for detection, such as a PCR or single-base extension reaction, assay components include at least one primer having a sequence complementary to a region adjacent to or encompassing such polymorphism. Likewise, for ligation-based detection assays, such as OLA, circularizable probe reactions, and the like, oligonucleotides are provided that are complementary to adjacent regions near, or encompassing, a polymorphism of the invention. In another embodiment, at least one allele-specific oligonucleotide is provided as described above. Often, the kits contain one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting at least one, or at least 5, or at least 10, or all of the polymorphisms shown in Table 1. Optional additional components of the kit include, for example, restriction enzymes, reverse transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions. Usually, the kit also contains instructions for carrying out the methods.

From the foregoing, it is apparent that the invention includes a number of general uses that can be expressed concisely as follows. The invention provides for the use of any of the nucleic acid segments described above in the diagnosis or monitoring of diseases, particularly CAD and related conditions. The invention further provides for the use of any of the nucleic acid segments in the manufacture of medicines for the treatment or prophylaxis of such diseases. The invention further provides for the use of any of the DNA segments as a pharmaceutical.

All publications and patent applications cited above are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

TABLE 1

| | | | | | | | | | | P-Value | P-Value | | | | | | | P-Value | P-Value | P-Value |
|---|---|---|---|---|---|---|---|---|---|---|---|---|---|---|---|---|---|---|---|---|
| | | | | Major | Minor | 1 | 2 | | | Allele | Allele | 1/1 | 1/2 | 2/2 | 1/1 | 1/2 | 2/2 | Genotype | Recessive | Dominant |
| SNP | Gene | Chrom | Posn | Allele | Allele | Case | Case | 1 Cntr | 2 Cntr | Chi\hat{ }2 | Fisher | Case | Case | Case | Cntr | Cntr | Cntr | Chi\hat{ }2 | Fisher | Fisher |
| rs12024382 | ENSG00000070831 | 1 | 22053947 | C | G | 599 | 29 | 580 | 48 | 0.02543 | 0.03364 | 285 | 29 | 0 | 268 | 44 | 2 | 0.060664 | 0.499203 | 0.048408 |
| rs6661161 | ENSG00000070831 | 1 | 22118977 | T | C | 509 | 117 | 536 | 90 | 0.03997 | 0.04774 | 204 | 101 | 8 | 229 | 78 | 6 | 0.096114 | 0.788147 | 0.037641 |
| rs16826506 | ENSG00000070831 | 1 | 22154596 | A | C | 599 | 25 | 619 | 7 | 0.00123 | 0.00113 | 288 | 23 | 1 | 306 | 7 | 0 | 0.006483 | 0.4992 | 0.001611 |
| rs2582020 | EPHB2 | 1 | 22852001 | C | T | 574 | 34 | 567 | 55 | 0.02782 | 0.036 | 270 | 34 | 0 | 258 | 51 | 2 | 0.061 | 0.499251 | 0.038057 |
| rs750012 | EPHB2 | 1 | 22855811 | C | T | 539 | 63 | 583 | 43 | 0.02488 | 0.02564 | 240 | 59 | 2 | 272 | 39 | 2 | 0.053682 | 1 | 0.022431 |
| rs10917325 | EPHB2 | 1 | 22954584 | C | T | 296 | 322 | 330 | 292 | 0.0693 | 0.07823 | 63 | 170 | 76 | 91 | 148 | 72 | 0.034828 | 0.706711 | 0.012032 |
| rs309476 | EPHB2 | 1 | 22958046 | A | C | 318 | 288 | 303 | 323 | 0.1529 | 0.15477 | 74 | 170 | 59 | 73 | 157 | 83 | 0.109767 | 0.044369 | 0.777132 |
| rs1467416 | EPHB2 | 1 | 22961345 | G | A | 338 | 290 | 311 | 317 | 0.12737 | 0.14205 | 83 | 172 | 59 | 78 | 155 | 81 | 0.105599 | 0.043837 | 0.714759 |
| rs1516526 | EPHB2 | 1 | 22968755 | A | G | 332 | 296 | 307 | 321 | 0.15823 | 0.17552 | 77 | 178 | 59 | 74 | 159 | 81 | 0.100864 | 0.043837 | 0.851903 |
| rs309499 | EPHB2 | 1 | 22980252 | C | T | 325 | 267 | 307 | 313 | 0.06078 | 0.06572 | 80 | 165 | 51 | 79 | 149 | 82 | 0.020986 | 0.007884 | 0.711979 |
| rs11591185 | AK2 | 1 | 33171277 | G | A | 547 | 81 | 569 | 59 | 0.04855 | 0.05941 | 239 | 69 | 6 | 256 | 57 | 1 | 0.070718 | 0.122907 | 0.117917 |
| rs6700177 | NFYC | 1 | 40887547 | G | A | 376 | 252 | 354 | 274 | 0.20831 | 0.22972 | 117 | 142 | 55 | 92 | 170 | 52 | 0.061195 | 0.831979 | 0.041984 |
| rs1289658 | TRIM45 | 1 | 117368130 | A | G | 379 | 249 | 353 | 275 | 0.1368 | 0.15252 | 109 | 161 | 44 | 104 | 145 | 65 | 0.082091 | 0.034765 | 0.736046 |
| rs7511654 | NOTCH2 | 1 | 120106754 | G | A | 618 | 4 | 622 | 0 | 0.04515 | 0.1244 | 307 | 4 | 0 | 311 | 0 | 0 | 0.133595 | 1 | 0.123793 |
| rs10923918 | NOTCH2 | 1 | 120124927 | G | T | 449 | 177 | 441 | 187 | 0.55785 | 0.5758 | 166 | 117 | 30 | 146 | 149 | 19 | 0.022377 | 0.104107 | 0.110375 |
| rs6685892 | NOTCH2 | 1 | 120170046 | A | T | 581 | 47 | 568 | 58 | 0.25489 | 0.26372 | 273 | 35 | 6 | 256 | 56 | 1 | 0.01132 | 0.122907 | 0.079385 |
| rs2793831 | NOTCH2 | 1 | 120235944 | T | C | 581 | 47 | 568 | 60 | 0.18886 | 0.22503 | 273 | 35 | 6 | 256 | 56 | 2 | 0.024817 | 0.28591 | 0.079385 |
| rs391102 | NOTCH2 | 1 | 120266176 | A | C | 580 | 48 | 566 | 62 | 0.16228 | 0.19423 | 272 | 36 | 6 | 254 | 58 | 2 | 0.0206 | 0.28591 | 0.065509 |
| rs12401436 | ENSG00000125462 | 1 | 153195543 | T | G | 454 | 170 | 418 | 210 | 0.01712 | 0.01945 | 169 | 116 | 27 | 146 | 126 | 42 | 0.069006 | 0.073483 | 0.055727 |
| rs716461 | PLXNA2 | 1 | 204646024 | G | A | 426 | 182 | 452 | 172 | 0.35804 | 0.37821 | 137 | 152 | 15 | 163 | 126 | 23 | 0.043579 | 0.24238 | 0.076818 |
| rs4844409 | PLXNA2 | 1 | 204677703 | C | T | 455 | 169 | 466 | 160 | 0.54063 | 0.56344 | 156 | 143 | 13 | 178 | 110 | 25 | 0.008475 | 0.064334 | 0.092401 |
| rs881758 | PLXNA2 | 1 | 204781943 | C | A | 473 | 153 | 496 | 132 | 0.14826 | 0.15725 | 173 | 127 | 13 | 201 | 94 | 19 | 0.017015 | 0.364499 | 0.028051 |
| rs10746422 | ENSG00000008141 | 1 | 206177098 | A | G | 423 | 205 | 390 | 238 | 0.05132 | 0.05874 | 145 | 133 | 36 | 117 | 156 | 41 | 0.076249 | 0.626731 | 0.028799 |
| rs742257 | ENSG00000008141 | 1 | 206183697 | C | T | 427 | 185 | 402 | 218 | 0.06502 | 0.06861 | 151 | 125 | 30 | 127 | 148 | 35 | 0.112562 | 0.600711 | 0.042883 |
| rs2072941 | ENSG00000008141 | 1 | 206199990 | T | C | 311 | 315 | 332 | 296 | 0.25911 | 0.28307 | 81 | 149 | 83 | 77 | 178 | 59 | 0.034598 | 0.022183 | 0.713636 |
| rs7519942 | ENSG00000008141 | 1 | 206214343 | G | C | 563 | 61 | 587 | 39 | 0.02087 | 0.02192 | 254 | 55 | 3 | 276 | 35 | 2 | 0.062161 | 0.685993 | 0.019497 |
| rs4675094 | IRS1 | 2 | 227477296 | G | C | 584 | 44 | 563 | 65 | 0.03531 | 0.04459 | 271 | 42 | 1 | 253 | 57 | 4 | 0.095797 | 0.373002 | 0.06765 |
| rs9879090 | ENSG00000163939 | 3 | 52623305 | C | T | 352 | 274 | 316 | 306 | 0.05465 | 0.06104 | 99 | 154 | 60 | 70 | 176 | 65 | 0.036213 | 0.617746 | 0.011633 |
| rs9877209 | MGLL | 3 | 128929337 | C | G | 337 | 291 | 372 | 256 | 0.04639 | 0.05296 | 88 | 161 | 65 | 109 | 154 | 51 | 0.129783 | 0.181132 | 0.085292 |
| rs6787155 | MGLL | 3 | 128946306 | A | C | 512 | 116 | 481 | 147 | 0.03157 | 0.03735 | 211 | 90 | 13 | 181 | 119 | 14 | 0.041651 | 1 | 0.016792 |
| rs608318 | MGLL | 3 | 128967455 | A | C | 551 | 77 | 536 | 92 | 0.21486 | 0.24696 | 245 | 61 | 8 | 225 | 86 | 3 | 0.025028 | 0.222431 | 0.080403 |
| MRD_3427 | SH3BP2 | 4 | 2943461 | T | G | 514 | 112 | 485 | 143 | 0.03184 | 0.03525 | 214 | 86 | 13 | 188 | 109 | 17 | 0.085172 | 0.575308 | 0.030373 |
| rs4961 | SH3BP2 | 4 | 2943715 | G | T | 515 | 113 | 487 | 141 | 0.04918 | 0.05772 | 214 | 87 | 13 | 190 | 107 | 17 | 0.133926 | 0.575308 | 0.055249 |
| rs2932965 | PPARGC1A | 4 | 23481755 | G | A | 476 | 152 | 509 | 119 | 0.0236 | 0.02804 | 182 | 112 | 20 | 204 | 101 | 9 | 0.049927 | 0.055524 | 0.084996 |
| rs3774921 | PPARGC1A | 4 | 23486916 | A | G | 330 | 298 | 355 | 273 | 0.15658 | 0.1738 | 92 | 146 | 76 | 94 | 167 | 53 | 0.062937 | 0.029528 | 0.93037 |
| rs3755863 | PPARGC1A | 4 | 23491791 | G | A | 371 | 257 | 347 | 281 | 0.17115 | 0.18967 | 115 | 141 | 58 | 87 | 173 | 54 | 0.026184 | 0.754598 | 0.020963 |
| rs2970853 | PPARGC1A | 4 | 23499788 | G | A | 444 | 180 | 482 | 146 | 0.02403 | 0.02438 | 160 | 124 | 28 | 183 | 116 | 15 | 0.056902 | 0.040733 | 0.09164 |
| rs6448227 | PPARGC1A | 4 | 23524900 | C | T | 482 | 146 | 514 | 112 | 0.01896 | 0.02109 | 185 | 112 | 17 | 210 | 94 | 9 | 0.060354 | 0.159717 | 0.03863 |
| rs1466414 | KLF3 | 4 | 38486890 | A | G | 360 | 182 | 431 | 161 | 0.0194 | 0.01996 | 121 | 118 | 32 | 157 | 117 | 22 | 0.06633 | 0.086169 | 0.053065 |
| rs337623 | KLF3 | 4 | 38496411 | C | T | 515 | 111 | 488 | 140 | 0.04354 | 0.04809 | 209 | 97 | 7 | 190 | 108 | 16 | 0.081465 | 0.087175 | 0.114781 |
| rs6531656 | KLF3 | 4 | 38506767 | T | C | 523 | 95 | 490 | 122 | 0.03583 | 0.03651 | 229 | 65 | 15 | 211 | 68 | 27 | 0.121355 | 0.05593 | 0.180009 |
| rs729064 | KLF3 | 4 | 38512489 | T | C | 500 | 88 | 477 | 121 | 0.01728 | 0.01819 | 228 | 44 | 22 | 209 | 59 | 31 | 0.105557 | 0.2503 | 0.040068 |
| rs3796533 | KLF3 | 4 | 38515372 | G | A | 544 | 84 | 506 | 122 | 0.00378 | 0.00473 | 237 | 70 | 7 | 208 | 90 | 16 | 0.019143 | 0.087175 | 0.01382 |
| rs4699733 | ENSG00000164025 | 4 | 100494712 | C | G | 427 | 201 | 460 | 168 | 0.04093 | 0.04737 | 144 | 139 | 31 | 170 | 120 | 24 | 0.10874 | 0.397193 | 0.045933 |
| rs1826909 | ENSG00000164025 | 4 | 100574921 | C | T | 398 | 230 | 371 | 257 | 0.11791 | 0.1321 | 122 | 154 | 38 | 115 | 141 | 58 | 0.084319 | 0.034731 | 0.621398 |
| rs1229984 | ENSG00000164025 | 4 | 100596497 | G | A | 602 | 26 | 584 | 44 | 0.02683 | 0.03585 | 288 | 26 | 0 | 275 | 34 | 5 | 0.041444 | 0.061506 | 0.115402 |
| rs17586163 | ENSG00000164025 | 4 | 100631749 | T | C | 562 | 66 | 585 | 43 | 0.02115 | 0.02707 | 253 | 56 | 5 | 273 | 39 | 2 | 0.078541 | 0.450496 | 0.039449 |
| rs10516440 | ENSG00000164025 | 4 | 100662571 | A | G | 541 | 85 | 570 | 58 | 0.01556 | 0.01647 | 231 | 79 | 3 | 259 | 52 | 3 | 0.02783 | 1 | 0.009115 |
| rs6851750 | ENSG00000164025 | 4 | 100670197 | C | T | 529 | 87 | 499 | 103 | 0.15099 | 0.15589 | 235 | 59 | 14 | 227 | 45 | 29 | 0.027651 | 0.017131 | 0.849887 |
| rs6532816 | ENSG00000164025 | 4 | 100675511 | C | T | 542 | 86 | 570 | 58 | 0.01315 | 0.01656 | 231 | 80 | 3 | 259 | 52 | 3 | 0.023059 | 1 | 0.009115 |
| rs991316 | ENSG00000164025 | 4 | 100679623 | G | A | 355 | 271 | 350 | 278 | 0.72739 | 0.73305 | 89 | 177 | 47 | 101 | 148 | 65 | 0.044226 | 0.076054 | 0.339305 |
| rs1154461 | ENSG00000164025 | 4 | 100700080 | G | C | 425 | 199 | 452 | 176 | 0.13543 | 0.13908 | 135 | 155 | 22 | 169 | 114 | 31 | 0.003068 | 0.250685 | 0.008459 |
| rs1573496 | ENSG00000164025 | 4 | 100706847 | C | G | 574 | 54 | 549 | 79 | 0.02187 | 0.02744 | 263 | 48 | 3 | 240 | 69 | 5 | 0.069916 | 0.724805 | 0.027639 |
| rs3811800 | MTP | 4 | 100851731 | A | G | 434 | 194 | 390 | 238 | 0.00896 | 0.0106 | 149 | 136 | 29 | 114 | 162 | 38 | 0.017118 | 0.30108 | 0.005911 |
| rs3816873 | MTP | 4 | 100861842 | T | C | 480 | 148 | 442 | 184 | 0.01938 | 0.02119 | 185 | 110 | 19 | 154 | 134 | 25 | 0.049488 | 0.353382 | 0.016241 |
| rs1057613 | MTP | 4 | 100862163 | G | A | 348 | 280 | 309 | 319 | 0.02758 | 0.03177 | 93 | 162 | 59 | 73 | 163 | 78 | 0.080148 | 0.081763 | 0.085381 |
| rs17599091 | MTP | 4 | 100870097 | C | G | 614 | 14 | 598 | 30 | 0.01407 | 0.02033 | 301 | 12 | 1 | 284 | 30 | 0 | 0.01001 | 1 | 0.010707 |
| rs2903202 | MTP | 4 | 100874183 | A | T | 564 | 62 | 539 | 89 | 0.02024 | 0.02385 | 255 | 54 | 4 | 229 | 81 | 4 | 0.033455 | 1 | 0.013188 |
| MRD_3470 | ENSG00000109471 | 4 | 123735585 | A | C | 424 | 202 | 452 | 174 | 0.08429 | 0.09592 | 140 | 144 | 29 | 170 | 112 | 31 | 0.030655 | 0.892126 | 0.020362 |
| rs6843773 | ENSG00000109436 | 4 | 141923723 | T | C | 321 | 293 | 348 | 276 | 0.21809 | 0.23117 | 88 | 145 | 74 | 91 | 166 | 55 | 0.120837 | 0.048529 | 0.929429 |
| rs4956472 | ENSG00000109436 | 4 | 141936979 | A | G | 335 | 291 | 373 | 255 | 0.03573 | 0.04029 | 92 | 151 | 70 | 106 | 161 | 47 | 0.054199 | 0.018543 | 0.264186 |
| rs6845527 | ENSG00000109436 | 4 | 141946906 | A | G | 303 | 325 | 340 | 288 | 0.03674 | 0.04209 | 71 | 161 | 82 | 92 | 156 | 66 | 0.104658 | 0.158305 | 0.068496 |
| rs1532013 | ENSG00000109436 | 4 | 142014072 | G | A | 341 | 287 | 297 | 331 | 0.01301 | 0.01519 | 95 | 151 | 68 | 71 | 155 | 88 | 0.047685 | 0.079118 | 0.037227 |
| rs4555724 | ENSG00000109436 | 4 | 142016302 | T | C | 379 | 247 | 340 | 288 | 0.02189 | 0.0225 | 114 | 151 | 48 | 97 | 146 | 71 | 0.052401 | 0.02481 | 0.151333 |
| rs6056 | FGA | 4 | 155846426 | C | T | 504 | 124 | 532 | 96 | 0.03766 | 0.04487 | 203 | 98 | 13 | 224 | 84 | 6 | 0.095912 | 0.16059 | 0.086991 |
| rs4220 | FGA | 4 | 155849364 | G | A | 504 | 124 | 534 | 94 | 0.02541 | 0.03057 | 203 | 98 | 13 | 225 | 84 | 5 | 0.056042 | 0.09151 | 0.071928 |
| rs765199 | NPR3 | 5 | 32778222 | G | A | 492 | 128 | 489 | 135 | 0.66913 | 0.67753 | 200 | 92 | 18 | 185 | 119 | 8 | 0.019456 | 0.046894 | 0.18731 |
| rs700926 | NPR3 | 5 | 32781040 | T | G | 493 | 135 | 468 | 160 | 0.09611 | 0.11007 | 198 | 97 | 19 | 170 | 128 | 16 | 0.035817 | 0.728431 | 0.028622 |
| rs6450502 | PDE4D | 5 | 58396235 | A | T | 611 | 17 | 597 | 31 | 0.03935 | 0.05465 | 297 | 17 | 0 | 284 | 29 | 1 | 0.109629 | 1 | 0.06781 |
| rs986067 | PDE4D | 5 | 58424151 | C | T | 611 | 17 | 597 | 31 | 0.03935 | 0.05465 | 297 | 17 | 0 | 284 | 29 | 1 | 0.109629 | 1 | 0.06781 |
| rs929820 | PDE4D | 5 | 58429089 | C | T | 436 | 192 | 416 | 212 | 0.227 | 0.25107 | 148 | 140 | 26 | 145 | 126 | 43 | 0.083912 | 0.040572 | 0.872917 |
| rs997421 | PDE4D | 5 | 58465399 | C | T | 580 | 48 | 561 | 67 | 0.06304 | 0.07785 | 272 | 36 | 6 | 249 | 63 | 2 | 0.005575 | 0.28591 | 0.019247 |
| rs1862563 | PDE4D | 5 | 58473284 | T | C | 535 | 93 | 496 | 130 | 0.0058 | 0.00626 | 229 | 77 | 8 | 197 | 102 | 14 | 0.023166 | 0.201555 | 0.00801 |
| rs1824788 | PDE4D | 5 | 58476015 | G | A | 342 | 286 | 307 | 321 | 0.04812 | 0.05484 | 96 | 150 | 68 | 75 | 157 | 82 | 0.132309 | 0.223668 | 0.072818 |
| rs6877256 | PDE4D | 5 | 58498498 | C | T | 594 | 32 | 577 | 51 | 0.0321 | 0.04035 | 282 | 30 | 1 | 266 | 45 | 3 | 0.107229 | 0.623801 | 0.053662 |
| rs9292201 | PDE4D | 5 | 58618370 | T | C | 536 | 92 | 561 | 67 | 0.03388 | 0.04143 | 227 | 82 | 5 | 250 | 61 | 3 | 0.095707 | 0.724805 | 0.03974 |
| rs921942 | PDE4D | 5 | 58823614 | C | A | 332 | 296 | 303 | 325 | 0.1017 | 0.11402 | 91 | 150 | 73 | 67 | 169 | 78 | 0.084467 | 0.708855 | 0.034229 |
| rs4551023 | PDE4D | 5 | 58837048 | C | T | 452 | 176 | 483 | 145 | 0.04492 | 0.0522 | 164 | 124 | 26 | 184 | 115 | 15 | 0.108633 | 0.105236 | 0.127106 |
| rs7728286 | PDE4D | 5 | 58854479 | C | T | 407 | 221 | 435 | 193 | 0.09282 | 0.10503 | 139 | 129 | 46 | 147 | 141 | 26 | 0.042582 | 0.016846 | 0.574905 |
| rs296410 | PDE4D | 5 | 58937990 | C | T | 295 | 333 | 334 | 294 | 0.02774 | 0.03195 | 67 | 161 | 86 | 91 | 152 | 71 | 0.06934 | 0.1969 | 0.034229 |
| rs12654264 | HMGCR | 5 | 74684359 | A | T | 395 | 233 | 399 | 229 | 0.81494 | 0.86066 | 127 | 141 | 46 | 115 | 169 | 30 | 0.03892 | 0.065921 | 0.367111 |
| rs3846663 | HMGCR | 5 | 74691482 | C | T | 395 | 233 | 402 | 226 | 0.68169 | 0.72518 | 127 | 141 | 46 | 116 | 170 | 28 | 0.02259 | 0.034817 | 0.412637 |
| rs252802 | EFNA5 | 5 | 106790483 | C | T | 305 | 323 | 338 | 290 | 0.06249 | 0.07082 | 78 | 149 | 87 | 88 | 162 | 64 | 0.097826 | 0.03974 | 0.415452 |
| rs152602 | EFNA5 | 5 | 106804017 | G | C | 430 | 196 | 393 | 235 | 0.02273 | 0.02392 | 145 | 140 | 28 | 122 | 149 | 43 | 0.066239 | 0.077115 | 0.063415 |
| rs152599 | EFNA5 | 5 | 106807204 | G | T | 403 | 225 | 362 | 266 | 0.01775 | 0.02068 | 124 | 155 | 35 | 105 | 152 | 57 | 0.03228 | 0.017443 | 0.135545 |
| rs537500 | EFNA5 | 5 | 106821490 | C | T | 461 | 167 | 488 | 138 | 0.06055 | 0.06536 | 168 | 125 | 21 | 194 | 100 | 19 | 0.093313 | 0.870439 | 0.03557 |
| rs3909931 | EFNA5 | 5 | 106821882 | T | A | 484 | 144 | 511 | 115 | 0.04614 | 0.05076 | 183 | 118 | 13 | 211 | 89 | 13 | 0.048532 | 1 | 0.020632 |
| rs152577 | EFNA5 | 5 | 106830196 | A | T | 383 | 245 | 377 | 249 | 0.782 | 0.81721 | 103 | 177 | 34 | 123 | 131 | 59 | 4.62E−04 | 0.004971 | 0.096568 |
| rs341276 | EFNA5 | 5 | 106898137 | C | G | 531 | 97 | 505 | 123 | 0.0536 | 0.06331 | 230 | 71 | 13 | 203 | 99 | 12 | 0.042101 | 1 | 0.024813 |
| rs180791 | EFNA5 | 5 | 106951468 | G | A | 537 | 89 | 555 | 73 | 0.17104 | 0.17866 | 233 | 71 | 9 | 243 | 69 | 2 | 0.09577 | 0.036788 | 0.401804 |
| rs352614 | EFNA5 | 5 | 106977080 | G | T | 493 | 135 | 526 | 102 | 0.01732 | 0.02088 | 195 | 103 | 16 | 221 | 84 | 9 | 0.063435 | 0.220073 | 0.034768 |
| rs3805665 | ENSG00000197208 | 5 | 131672617 | G | A | 593 | 35 | 569 | 57 | 0.01646 | 0.01726 | 279 | 35 | 0 | 261 | 47 | 5 | 0.025292 | 0.030506 | 0.050206 |
| rs270606 | ENSG00000197208 | 5 | 131678766 | C | T | 417 | 205 | 447 | 181 | 0.11347 | 0.12584 | 135 | 147 | 29 | 164 | 119 | 31 | 0.054681 | 0.892199 | 0.030634 |
| rs156322 | ENSG00000197208 | 5 | 131681824 | A | G | 415 | 207 | 447 | 181 | 0.0885 | 0.09876 | 133 | 149 | 29 | 164 | 119 | 31 | 0.036039 | 0.892199 | 0.020184 |
| rs273915 | ENSG00000197208 | 5 | 131688018 | G | C | 419 | 209 | 447 | 181 | 0.08773 | 0.09959 | 134 | 151 | 29 | 164 | 119 | 31 | 0.032074 | 0.892144 | 0.020405 |
| rs3806837 | PCDHGA2 | 5 | 140703358 | A | G | 573 | 55 | 548 | 80 | 0.02275 | 0.02848 | 260 | 53 | 1 | 238 | 72 | 4 | 0.059016 | 0.373002 | 0.03835 |
| rs11575948 | PCDHGA2 | 5 | 140704244 | A | C | 589 | 39 | 567 | 61 | 0.02184 | 0.02817 | 275 | 39 | 0 | 255 | 57 | 2 | 0.04666 | 0.499203 | 0.036301 |
| rs6877775 | PCDHGA2 | 5 | 140707976 | G | A | 589 | 39 | 566 | 60 | 0.02671 | 0.02805 | 275 | 39 | 0 | 255 | 56 | 2 | 0.055154 | 0.248804 | 0.036301 |
| rs10214288 | PCDHGA2 | 5 | 140714062 | A | G | 589 | 39 | 567 | 59 | 0.03395 | 0.03563 | 275 | 39 | 0 | 256 | 55 | 2 | 0.067149 | 0.248804 | 0.046458 |
| rs4151698 | PCDHGA2 | 5 | 140733429 | A | G | 580 | 42 | 561 | 63 | 0.03363 | 0.04099 | 269 | 42 | 0 | 251 | 59 | 2 | 0.064478 | 0.499197 | 0.051886 |
| rs3806832 | PCDHGA2 | 5 | 140745907 | C | T | 604 | 22 | 588 | 40 | 0.01971 | 0.02608 | 291 | 22 | 0 | 274 | 40 | 0 | 0.05682 | 1 | 0.022325 |
| rs17208397 | PCDHGA2 | 5 | 140778823 | G | C | 508 | 116 | 555 | 73 | 5.77E−04 | 6.63E−04 | 211 | 86 | 15 | 244 | 67 | 3 | 0.001706 | 0.003826 | 0.005379 |
| rs3805695 | PCDHGA2 | 5 | 140838429 | G | A | 539 | 83 | 518 | 110 | 0.04125 | 0.04216 | 232 | 75 | 4 | 212 | 94 | 8 | 0.113266 | 0.383074 | 0.052892 |
| rs6865659 | CSF1R-PDGFRB | 5 | 149465965 | A | G | 402 | 216 | 445 | 183 | 0.02793 | 0.02894 | 135 | 132 | 42 | 152 | 141 | 21 | 0.01605 | 0.005028 | 0.260579 |
| rs165979 | CSF1R-PDGFRB | 5 | 149475963 | G | A | 304 | 286 | 353 | 269 | 0.06792 | 0.07381 | 85 | 134 | 76 | 94 | 165 | 52 | 0.020759 | 0.007156 | 0.722252 |
| rs1075846 | CSF1R-PDGFRB | 5 | 149484351 | A | G | 397 | 211 | 424 | 200 | 0.32351 | 0.33399 | 141 | 115 | 48 | 141 | 142 | 29 | 0.02445 | 0.020153 | 0.808384 |
| rs6579775 | CSF1R-PDGFRB | 5 | 149514041 | C | T | 512 | 112 | 487 | 141 | 0.04724 | 0.04899 | 209 | 94 | 9 | 190 | 107 | 17 | 0.122411 | 0.159746 | 0.09687 |
| MRD_3435 | KCNMB1 | 5 | 169748952 | C | T | 540 | 44 | 589 | 29 | 0.03924 | 0.04065 | 249 | 42 | 1 | 280 | 29 | 0 | 0.094428 | 0.485857 | 0.045369 |
| rs2804921 | ENSG00000124523 | 6 | 13702645 | G | T | 475 | 151 | 445 | 183 | 0.04441 | 0.0477 | 182 | 111 | 20 | 159 | 127 | 28 | 0.138159 | 0.293049 | 0.065249 |
| rs1052215 | ZNF305 | 6 | 28456137 | C | A | 416 | 212 | 381 | 247 | 0.04028 | 0.04629 | 138 | 140 | 36 | 120 | 141 | 53 | 0.105054 | 0.066703 | 0.167883 |
| MRD_3459 | AIF1 | 6 | 31691910 | C | T | 538 | 90 | 565 | 63 | 0.01984 | 0.02465 | 230 | 78 | 6 | 255 | 55 | 4 | 0.058833 | 0.751925 | 0.022183 |
| rs1035798 | AGPAT1-PBX2- | 6 | 32259200 | C | T | 497 | 127 | 463 | 165 | 0.01324 | 0.01351 | 199 | 99 | 14 | 173 | 117 | 24 | 0.05124 | 0.131096 | 0.028222 |
| | NOTCH4 | | | | | | | | | | | | | | | | | | | |
| rs2071280 | AGPAT1-PBX2- | 6 | 32272847 | C | G | 570 | 22 | 561 | 39 | 0.0292 | 0.03493 | 277 | 16 | 3 | 261 | 39 | 0 | 0.001453 | 0.121874 | 0.008268 |
| | NOTCH4 | | | | | | | | | | | | | | | | | | | |
| rs2071287 | AGPAT1-PBX2- | 6 | 32278411 | G | A | 338 | 284 | 302 | 326 | 0.02704 | 0.0275 | 94 | 150 | 67 | 78 | 146 | 90 | 0.086399 | 0.042752 | 0.151787 |
| | NOTCH4 | | | | | | | | | | | | | | | | | | | |
| rs2071286 | AGPAT1-PBX2- | 6 | 32287874 | G | A | 519 | 105 | 488 | 136 | 0.02621 | 0.03131 | 218 | 83 | 11 | 196 | 96 | 20 | 0.094132 | 0.139391 | 0.075098 |
| | NOTCH4 | | | | | | | | | | | | | | | | | | | |
| rs3131290 | AGPAT1-PBX2- | 6 | 32291153 | G | A | 402 | 224 | 364 | 264 | 0.0231 | 0.02399 | 128 | 146 | 39 | 114 | 136 | 64 | 0.026905 | 0.009461 | 0.251346 |
| | NOTCH4 | | | | | | | | | | | | | | | | | | | |
| rs423023 | AGPAT1-PBX2- | 6 | 32296275 | C | G | 444 | 174 | 409 | 217 | 0.01341 | 0.01456 | 163 | 118 | 28 | 136 | 137 | 40 | 0.05115 | 0.157781 | 0.024557 |
| | NOTCH4 | | | | | | | | | | | | | | | | | | | |
| rs422951 | AGPAT1-PBX2- | 6 | 32296361 | A | G | 357 | 253 | 327 | 295 | 0.03556 | 0.03901 | 106 | 145 | 54 | 93 | 141 | 77 | 0.086927 | 0.038466 | 0.227676 |
| | NOTCH4 | | | | | | | | | | | | | | | | | | | |
| rs520803 | AGPAT1-PBX2- | 6 | 32296581 | G | A | 440 | 174 | 407 | 217 | 0.01485 | 0.01705 | 161 | 118 | 28 | 135 | 137 | 40 | 0.055657 | 0.158035 | 0.024349 |
| | NOTCH4 | | | | | | | | | | | | | | | | | | | |
| rs415929 | AGPAT1-PBX2- | 6 | 32297010 | A | G | 440 | 174 | 409 | 217 | 0.01653 | 0.01721 | 161 | 118 | 28 | 136 | 137 | 40 | 0.061416 | 0.158526 | 0.02988 |
| | NOTCH4 | | | | | | | | | | | | | | | | | | | |
| rs443198 | AGPAT1-PBX2- | 6 | 32298384 | T | C | 375 | 243 | 360 | 264 | 0.28418 | 0.29888 | 107 | 161 | 41 | 109 | 142 | 61 | 0.077416 | 0.039513 | 1 |
| | NOTCH4 | | | | | | | | | | | | | | | | | | | |
| rs3134931 | AGPAT1-PBX2- | 6 | 32298598 | A | G | 428 | 188 | 398 | 226 | 0.03338 | 0.03515 | 152 | 124 | 32 | 126 | 146 | 40 | 0.07857 | 0.381143 | 0.029167 |
| | NOTCH4 | | | | | | | | | | | | | | | | | | | |
| rs1062193 | PTP4A1 | 6 | 64350112 | A | G | 617 | 7 | 611 | 17 | 0.04083 | 0.06143 | 305 | 7 | 0 | 297 | 17 | 0 | 0.118444 | 1 | 0.058927 |
| rs1597555 | ENSG00000105778 | 7 | 32319358 | A | C | 388 | 240 | 360 | 268 | 0.10744 | 0.12055 | 120 | 148 | 46 | 93 | 174 | 47 | 0.062892 | 1 | 0.0283 |
| rs7806941 | ENSG00000105778 | 7 | 32326559 | G | T | 388 | 240 | 360 | 268 | 0.10744 | 0.12055 | 120 | 148 | 46 | 93 | 174 | 47 | 0.062892 | 1 | 0.0283 |
| rs10951338 | ENSG00000105778 | 7 | 32333329 | A | G | 377 | 233 | 353 | 265 | 0.09466 | 0.10365 | 117 | 143 | 45 | 91 | 171 | 47 | 0.056009 | 0.910239 | 0.021405 |
| rs17365780 | ENSG00000105778 | 7 | 32402641 | T | C | 499 | 127 | 537 | 91 | 0.00676 | 0.00728 | 199 | 101 | 13 | 229 | 79 | 6 | 0.025109 | 0.109528 | 0.012894 |
| rs6966960 | ENSG00000105778 | 7 | 32639711 | A | T | 444 | 184 | 482 | 146 | 0.01484 | 0.01761 | 160 | 124 | 30 | 185 | 112 | 17 | 0.049353 | 0.06781 | 0.054163 |
| rs17150806 | ENSG00000105778 | 7 | 32819284 | C | T | 502 | 124 | 531 | 97 | 0.04264 | 0.04546 | 199 | 104 | 10 | 224 | 83 | 7 | 0.112835 | 0.474189 | 0.040979 |
| rs1534313 | SEMA3C | 7 | 80015555 | T | C | 543 | 85 | 561 | 67 | 0.11941 | 0.14116 | 237 | 69 | 8 | 247 | 67 | 0 | 0.016277 | 0.007468 | 0.392978 |
| rs979856 | SEMA3C | 7 | 80183857 | G | C | 417 | 211 | 382 | 246 | 0.0401 | 0.04609 | 144 | 129 | 41 | 124 | 134 | 56 | 0.141764 | 0.121816 | 0.125227 |
| rs7793260 | CALCR | 7 | 92696781 | C | T | 463 | 165 | 429 | 199 | 0.03446 | 0.04005 | 165 | 133 | 16 | 142 | 145 | 27 | 0.079856 | 0.113191 | 0.078967 |
| rs10243420 | CALCR | 7 | 92710824 | G | A | 394 | 234 | 422 | 202 | 0.06942 | 0.07515 | 121 | 152 | 41 | 146 | 130 | 36 | 0.112171 | 0.626809 | 0.043269 |
| rs17165475 | CALCR | 7 | 92728359 | C | T | 628 | 0 | 624 | 4 | 0.04516 | 0.1244 | 314 | 0 | 0 | 310 | 4 | 0 | 0.133611 | 1 | 0.123805 |
| rs8491 | PON1 | 7 | 94570265 | C | A | 404 | 222 | 355 | 269 | 0.00565 | 0.00647 | 123 | 158 | 32 | 99 | 157 | 56 | 0.010349 | 0.005789 | 0.054546 |
| rs757158 | PON1 | 7 | 94600179 | C | T | 341 | 285 | 371 | 257 | 0.09989 | 0.11043 | 83 | 175 | 55 | 119 | 133 | 62 | 0.001873 | 0.538723 | 0.002744 |
| rs7778571 | ZNF3 | 7 | 99309468 | A | G | 489 | 139 | 489 | 135 | 0.83088 | 0.83784 | 182 | 125 | 7 | 194 | 101 | 17 | 0.02884 | 0.038914 | 0.289758 |
| rs6592 | ZNF3 | 7 | 99312758 | C | A | 456 | 166 | 423 | 201 | 0.03247 | 0.03464 | 171 | 114 | 26 | 144 | 135 | 33 | 0.085679 | 0.411892 | 0.030544 |
| rs6960432 | ZNF3 | 7 | 99315526 | C | G | 363 | 265 | 383 | 245 | 0.2505 | 0.27497 | 96 | 171 | 47 | 121 | 141 | 52 | 0.049358 | 0.661529 | 0.043897 |
| rs264 | LPL | 8 | 19857460 | G | A | 544 | 80 | 524 | 104 | 0.06166 | 0.06647 | 243 | 58 | 11 | 219 | 86 | 9 | 0.031987 | 0.657621 | 0.023052 |
| rs10503814 | CLU | 8 | 27510492 | C | T | 605 | 19 | 595 | 33 | 0.05005 | 0.06468 | 294 | 17 | 1 | 281 | 33 | 0 | 0.040608 | 0.498403 | 0.039968 |
| rs3824260 | CYP7A1 | 8 | 59575744 | G | A | 366 | 262 | 411 | 217 | 0.00895 | 0.01055 | 112 | 142 | 60 | 138 | 135 | 41 | 0.039653 | 0.05018 | 0.04145 |
| rs7833904 | CYP7A1 | 8 | 59580216 | T | A | 358 | 270 | 394 | 234 | 0.03823 | 0.04387 | 95 | 168 | 51 | 125 | 144 | 45 | 0.042594 | 0.579444 | 0.015187 |
| rs9643665 | STAU2 | 8 | 74609639 | C | G | 550 | 78 | 546 | 82 | 0.73497 | 0.79965 | 238 | 74 | 2 | 243 | 60 | 11 | 0.020802 | 0.021141 | 0.706278 |
| rs1454627 | VLDLR | 9 | 2621738 | A | G | 400 | 228 | 440 | 186 | 0.01305 | 0.01384 | 131 | 138 | 45 | 157 | 126 | 30 | 0.052573 | 0.084409 | 0.03729 |
| rs1551411 | VLDLR | 9 | 2621932 | C | T | 453 | 175 | 488 | 140 | 0.02271 | 0.02679 | 164 | 125 | 25 | 191 | 106 | 17 | 0.076532 | 0.263318 | 0.036265 |
| rs10812381 | VLDLR | 9 | 2624646 | T | A | 332 | 296 | 309 | 319 | 0.1942 | 0.21429 | 95 | 142 | 77 | 68 | 173 | 73 | 0.022039 | 0.778958 | 0.017795 |
| rs1329024 | ELAVL2 | 9 | 23696348 | A | G | 550 | 44 | 536 | 68 | 0.02207 | 0.02268 | 264 | 22 | 11 | 249 | 38 | 15 | 0.071387 | 0.548676 | 0.026933 |
| rs7038525 | ELAVL2 | 9 | 23762277 | C | T | 507 | 121 | 533 | 95 | 0.05188 | 0.0614 | 204 | 99 | 11 | 228 | 77 | 9 | 0.117459 | 0.820931 | 0.047472 |
| rs9410448 | ENSG00000148082 | 9 | 88875259 | A | G | 372 | 256 | 339 | 289 | 0.06027 | 0.06843 | 103 | 166 | 45 | 96 | 147 | 71 | 0.026955 | 0.009943 | 0.606885 |
| rs3750399 | ENSG00000148082 | 9 | 88886517 | C | T | 412 | 216 | 394 | 234 | 0.28949 | 0.31712 | 126 | 160 | 28 | 125 | 144 | 45 | 0.090492 | 0.045787 | 1 |
| rs3758294 | ABCA1 | 9 | 104744370 | T | C | 537 | 87 | 490 | 138 | 2.15E−04 | 2.27E−04 | 227 | 83 | 2 | 193 | 104 | 17 | 2.09E−04 | 6.12E−04 | 0.002908 |
| rs2575878 | ABCA1 | 9 | 104746634 | C | T | 320 | 306 | 354 | 274 | 0.06221 | 0.06988 | 81 | 158 | 74 | 105 | 144 | 65 | 0.114926 | 0.388253 | 0.044255 |
| rs4149265 | ABCA1 | 9 | 104752053 | C | T | 488 | 140 | 447 | 179 | 0.01041 | 0.01142 | 187 | 114 | 13 | 162 | 123 | 28 | 0.022161 | 0.015668 | 0.053837 |
| rs5951621 | SMPX | X | 21488620 | A | C | 501 | 125 | 540 | 86 | 0.00324 | 0.00404 | 234 | 33 | 46 | 258 | 24 | 31 | 0.063486 | 0.087938 | 0.024784 |
| rs3788756 | SMPX | X | 21522578 | A | C | 359 | 269 | 395 | 233 | 0.0381 | 0.04373 | 156 | 47 | 111 | 173 | 49 | 92 | 0.259446 | 0.124503 | 0.201071 |
| rs6633442 | SMPX | X | 21534457 | A | G | 306 | 322 | 347 | 281 | 0.02058 | 0.02384 | 129 | 48 | 137 | 151 | 45 | 118 | 0.197797 | 0.143506 | 0.091733 |
| rs11544223 | FGD1 | X | 54354049 | A | G | 618 | 8 | 607 | 21 | 0.01494 | 0.02254 | 306 | 6 | 1 | 298 | 11 | 5 | 0.119934 | 0.216504 | 0.087175 |
| rs12011120 | FGD1 | X | 54359170 | A | G | 606 | 8 | 603 | 21 | 0.01645 | 0.02264 | 300 | 6 | 1 | 296 | 11 | 5 | 0.1272 | 0.216563 | 0.087333 |
| rs1155955 | OPHN1 | X | 67080112 | G | A | 593 | 33 | 577 | 51 | 0.04358 | 0.05428 | 290 | 13 | 10 | 281 | 15 | 18 | 0.276811 | 0.174992 | 0.207289 |
| rs5965536 | OPHN1 | X | 67300796 | G | C | 542 | 86 | 565 | 63 | 0.04475 | 0.0546 | 261 | 20 | 33 | 274 | 17 | 23 | 0.309616 | 0.207289 | 0.177378 |
| rs10752256 | SEC61A2 | 10 | 12213178 | A | G | 328 | 290 | 356 | 266 | 0.14079 | 0.15342 | 91 | 146 | 72 | 97 | 162 | 52 | 0.119908 | 0.044743 | 0.662715 |
| rs7919751 | SEC61A2 | 10 | 12226455 | A | G | 336 | 292 | 308 | 318 | 0.12752 | 0.14181 | 101 | 134 | 79 | 75 | 158 | 80 | 0.054524 | 0.927054 | 0.026181 |
| rs1441017 | PARD3 | 10 | 34475456 | C | A | 398 | 230 | 436 | 192 | 0.0232 | 0.02702 | 126 | 146 | 42 | 148 | 140 | 26 | 0.059101 | 0.053428 | 0.090991 |
| rs1660619 | PARD3 | 10 | 34491115 | C | T | 385 | 243 | 425 | 203 | 0.01835 | 0.02142 | 119 | 147 | 48 | 151 | 123 | 40 | 0.035915 | 0.421109 | 0.012398 |
| rs1274484 | PARD3 | 10 | 34496697 | A | G | 379 | 249 | 344 | 284 | 0.0457 | 0.0522 | 116 | 147 | 51 | 91 | 162 | 61 | 0.098257 | 0.348183 | 0.041485 |
| rs1274474 | PARD3 | 10 | 34519660 | G | C | 299 | 325 | 339 | 289 | 0.03186 | 0.03642 | 76 | 147 | 89 | 85 | 169 | 60 | 0.021572 | 0.006445 | 0.465007 |
| rs1274478 | PARD3 | 10 | 34527008 | G | A | 370 | 254 | 423 | 201 | 0.00183 | 0.00221 | 106 | 158 | 48 | 135 | 153 | 24 | 0.003073 | 0.003711 | 0.021233 |
| rs648768 | PARD3 | 10 | 34530607 | A | G | 428 | 200 | 389 | 239 | 0.021 | 0.02448 | 144 | 140 | 30 | 113 | 163 | 38 | 0.040229 | 0.368787 | 0.014829 |
| rs1121682 | PARD3 | 10 | 34532194 | C | A | 434 | 192 | 486 | 142 | 0.00125 | 0.00139 | 151 | 132 | 30 | 188 | 110 | 16 | 0.005806 | 0.032785 | 0.003912 |
| rs2570326 | PARD3 | 10 | 34553836 | G | A | 310 | 318 | 341 | 287 | 0.08001 | 0.0902 | 81 | 148 | 85 | 89 | 163 | 62 | 0.095437 | 0.037922 | 0.529637 |
| rs1545214 | PARD3 | 10 | 34560365 | C | A | 444 | 184 | 502 | 126 | 1.47E−04 | 1.85E−04 | 160 | 124 | 30 | 197 | 108 | 9 | 2.97E−04 | 7.37E−04 | 0.00369 |
| rs1111267 | PARD3 | 10 | 34567393 | C | A | 462 | 166 | 494 | 132 | 0.02613 | 0.02855 | 171 | 120 | 23 | 194 | 106 | 13 | 0.078365 | 0.121303 | 0.062606 |
| rs2570322 | PARD3 | 10 | 34580172 | G | A | 586 | 42 | 611 | 17 | 8.56E−04 | 0.00119 | 273 | 40 | 1 | 298 | 15 | 1 | 0.001971 | 1 | 7.32E−04 |
| rs1570650 | PARD3 | 10 | 34970874 | G | A | 615 | 5 | 628 | 0 | 0.02414 | 0.03002 | 305 | 5 | 0 | 314 | 0 | 0 | 0.07787 | 1 | 0.02977 |
| rs16912153 | UBE2D1 | 10 | 59765272 | G | A | 603 | 21 | 588 | 36 | 0.04197 | 0.0568 | 291 | 21 | 0 | 277 | 34 | 1 | 0.109822 | 1 | 0.067839 |
| rs10509089 | UBE2D1 | 10 | 59776243 | A | T | 621 | 7 | 609 | 19 | 0.0174 | 0.02734 | 307 | 7 | 0 | 295 | 19 | 0 | 0.055641 | 1 | 0.025756 |
| rs7907725 | UBE2D1 | 10 | 59781919 | A | G | 621 | 7 | 609 | 19 | 0.0174 | 0.02734 | 307 | 7 | 0 | 295 | 19 | 0 | 0.055641 | 1 | 0.025756 |
| rs10826174 | UBE2D1 | 10 | 59797416 | G | C | 387 | 241 | 352 | 276 | 0.04478 | 0.0512 | 124 | 139 | 51 | 92 | 168 | 54 | 0.022756 | 0.830733 | 0.009133 |
| rs10826175 | UBE2D1 | 10 | 59801062 | G | A | 365 | 263 | 328 | 300 | 0.03579 | 0.03604 | 110 | 145 | 59 | 77 | 174 | 63 | 0.013629 | 0.762303 | 0.005158 |
| rs2763345 | ENSG00000099288 | 10 | 71273522 | G | C | 580 | 48 | 556 | 72 | 0.02124 | 0.02692 | 267 | 46 | 1 | 249 | 58 | 7 | 0.038532 | 0.068572 | 0.076054 |
| rs2763356 | ENSG00000099288 | 10 | 71298608 | T | G | 590 | 10 | 603 | 21 | 0.05863 | 0.06888 | 291 | 8 | 1 | 291 | 21 | 0 | 0.036979 | 0.490196 | 0.038964 |
| rs10768219 | RAG1 | 11 | 36579047 | C | T | 391 | 237 | 353 | 275 | 0.02911 | 0.03357 | 124 | 143 | 47 | 109 | 135 | 70 | 0.057348 | 0.023871 | 0.247466 |
| rs3824894 | ENSG00000175274 | 11 | 44892047 | A | G | 347 | 275 | 308 | 318 | 0.01982 | 0.02025 | 104 | 139 | 68 | 74 | 160 | 79 | 0.025376 | 0.345996 | 0.00777 |
| rs835984 | ENSG00000175274 | 11 | 44892269 | G | A | 328 | 264 | 378 | 238 | 0.03567 | 0.04093 | 88 | 152 | 56 | 114 | 150 | 44 | 0.102122 | 0.15437 | 0.069934 |
| rs4945138 | CAPN5 | 11 | 76457861 | G | A | 344 | 258 | 335 | 287 | 0.24775 | 0.25062 | 107 | 130 | 64 | 85 | 165 | 61 | 0.037184 | 0.617794 | 0.029718 |
| rs7926553 | CAPN5 | 11 | 76470079 | A | C | 378 | 242 | 413 | 213 | 0.06647 | 0.0684 | 120 | 138 | 52 | 132 | 149 | 32 | 0.056681 | 0.018863 | 0.414412 |
| rs614128 | NOX4 | 11 | 88853762 | G | C | 580 | 48 | 604 | 24 | 0.00358 | 0.00492 | 266 | 48 | 0 | 291 | 22 | 1 | 0.002768 | 1 | 0.002303 |
| rs490934 | NOX4 | 11 | 88863264 | G | C | 556 | 48 | 587 | 23 | 0.00193 | 0.0021 | 254 | 48 | 0 | 283 | 21 | 1 | 0.001418 | 1 | 8.88E−04 |
| MRD_3565 | NOX4 | 11 | 88864101 | C | A | 372 | 134 | 401 | 105 | 0.03185 | 0.03219 | 131 | 110 | 12 | 159 | 83 | 11 | 0.038309 | 1 | 0.015148 |
| rs553635 | NOX4 | 11 | 88869079 | C | T | 575 | 51 | 594 | 32 | 0.03091 | 0.04032 | 263 | 49 | 1 | 282 | 30 | 1 | 0.073094 | 1 | 0.031588 |
| rs7914 | MCAM | 11 | 118685526 | C | T | 469 | 127 | 442 | 168 | 0.01183 | 0.01314 | 182 | 105 | 11 | 154 | 134 | 17 | 0.029341 | 0.334334 | 0.01097 |
| rs2846690 | ENSG00000120471 | 11 | 128314137 | G | A | 424 | 172 | 478 | 144 | 0.0231 | 0.02614 | 154 | 116 | 28 | 189 | 100 | 22 | 0.074219 | 0.305783 | 0.027323 |
| rs17123579 | ENSG00000123416 | 12 | 47808845 | A | G | 396 | 228 | 439 | 189 | 0.01558 | 0.01649 | 123 | 150 | 39 | 158 | 123 | 33 | 0.023244 | 0.454525 | 0.006381 |
| rs177079 | ENSG00000074729 | 12 | 51172381 | G | A | 376 | 236 | 411 | 211 | 0.09003 | 0.09727 | 109 | 158 | 39 | 139 | 133 | 39 | 0.056794 | 1 | 0.026525 |
| rs941022 | BLOC1S1 | 12 | 54397591 | T | G | 368 | 256 | 336 | 292 | 0.05105 | 0.05293 | 112 | 144 | 56 | 77 | 182 | 55 | 0.004267 | 0.916886 | 0.00228 |
| rs1513103 | PTPRR | 12 | 69388653 | G | A | 525 | 103 | 552 | 76 | 0.02931 | 0.03563 | 223 | 79 | 12 | 245 | 62 | 7 | 0.110824 | 0.351819 | 0.054264 |
| rs1513099 | PTPRR | 12 | 69437900 | A | G | 425 | 203 | 388 | 240 | 0.02889 | 0.03345 | 145 | 135 | 34 | 116 | 156 | 42 | 0.061428 | 0.391866 | 0.023295 |
| rs10506608 | PTPRR | 12 | 69441647 | A | G | 561 | 67 | 584 | 44 | 0.02223 | 0.02836 | 250 | 61 | 3 | 274 | 36 | 4 | 0.021436 | 1 | 0.013279 |
| rs9971690 | CRADD | 12 | 92606492 | G | A | 331 | 295 | 313 | 315 | 0.28237 | 0.28378 | 94 | 143 | 76 | 71 | 171 | 72 | 0.054765 | 0.707583 | 0.037227 |
| rs7304935 | CRADD | 12 | 92723753 | T | C | 447 | 179 | 399 | 229 | 0.00293 | 0.00314 | 153 | 141 | 19 | 129 | 141 | 44 | 0.002527 | 0.001278 | 0.054163 |
| rs1485888 | CRADD | 12 | 92728005 | A | C | 380 | 246 | 416 | 212 | 0.04166 | 0.04612 | 120 | 140 | 53 | 137 | 142 | 35 | 0.089863 | 0.039001 | 0.194066 |
| rs10859600 | CRADD | 12 | 92742628 | T | C | 391 | 235 | 430 | 198 | 0.02519 | 0.02795 | 127 | 137 | 49 | 150 | 130 | 34 | 0.090603 | 0.078202 | 0.076955 |
| rs1165679 | TRA1 | 12 | 102825705 | C | A | 497 | 105 | 531 | 81 | 0.0419 | 0.0463 | 205 | 87 | 9 | 229 | 73 | 4 | 0.108915 | 0.171777 | 0.072339 |
| rs1177457 | TRA1 | 12 | 102838594 | C | T | 450 | 176 | 407 | 221 | 0.00707 | 0.00756 | 160 | 130 | 23 | 128 | 151 | 35 | 0.022302 | 0.128915 | 0.010349 |
| rs6486602 | EPIM | 12 | 129831194 | G | C | 373 | 253 | 406 | 222 | 0.06449 | 0.0711 | 106 | 161 | 46 | 120 | 166 | 28 | 0.069931 | 0.026248 | 0.279761 |
| rs7962097 | EPIM | 12 | 129849744 | G | A | 499 | 119 | 524 | 102 | 0.17179 | 0.18211 | 205 | 89 | 15 | 217 | 90 | 6 | 0.123785 | 0.04738 | 0.440631 |
| rs368392 | ENSG00000073910 | 13 | 31539978 | C | T | 484 | 144 | 494 | 132 | 0.43075 | 0.45365 | 192 | 100 | 22 | 189 | 116 | 8 | 0.020852 | 0.013723 | 0.870239 |
| rs733258 | ENSG00000073910 | 13 | 31554200 | T | C | 520 | 108 | 492 | 136 | 0.04583 | 0.054 | 222 | 76 | 16 | 194 | 104 | 16 | 0.044153 | 1 | 0.022592 |
| rs17077090 | ENSG00000073910 | 13 | 31586074 | C | T | 615 | 13 | 603 | 25 | 0.04807 | 0.06865 | 302 | 11 | 1 | 289 | 25 | 0 | 0.034555 | 1 | 0.040687 |
| rs12877839 | ENSG00000073910 | 13 | 31745833 | T | A | 401 | 213 | 372 | 250 | 0.04569 | 0.04602 | 133 | 135 | 39 | 121 | 130 | 60 | 0.078468 | 0.0283 | 0.288037 |
| rs17255807 | ENSG00000050130 | 14 | 59026912 | G | A | 601 | 25 | 587 | 41 | 0.04442 | 0.05711 | 290 | 21 | 2 | 274 | 39 | 1 | 0.045373 | 0.623801 | 0.032893 |
| rs743165 | ENSG00000050130 | 14 | 59041958 | T | C | 359 | 269 | 322 | 304 | 0.04176 | 0.0472 | 111 | 137 | 66 | 84 | 154 | 75 | 0.070493 | 0.390642 | 0.024813 |
| rs716218 | MEIS2 | 15 | 34979253 | G | A | 375 | 253 | 389 | 239 | 0.41836 | 0.45239 | 105 | 165 | 44 | 130 | 129 | 55 | 0.015844 | 0.273442 | 0.047699 |
| rs3099886 | MEIS2 | 15 | 35049404 | T | C | 487 | 141 | 461 | 167 | 0.08815 | 0.10098 | 181 | 125 | 8 | 173 | 115 | 26 | 0.006324 | 0.002247 | 0.573303 |
| rs1357470 | MEIS2 | 15 | 35067343 | G | C | 493 | 135 | 462 | 166 | 0.04045 | 0.04726 | 187 | 119 | 8 | 174 | 114 | 26 | 0.006394 | 0.002247 | 0.332742 |
| rs10518924 | MEIS2 | 15 | 35088857 | A | G | 577 | 51 | 556 | 72 | 0.04619 | 0.05724 | 266 | 45 | 3 | 248 | 60 | 6 | 0.151586 | 0.50465 | 0.07811 |
| rs10518928 | MEIS2 | 15 | 35140532 | T | C | 501 | 127 | 487 | 141 | 0.33493 | 0.37062 | 208 | 85 | 21 | 184 | 119 | 11 | 0.005913 | 0.101097 | 0.057984 |
| rs4625687 | MEIS2 | 15 | 35151546 | A | G | 440 | 188 | 405 | 223 | 0.03531 | 0.04082 | 155 | 130 | 29 | 123 | 159 | 32 | 0.034372 | 0.787768 | 0.012692 |
| rs4244559 | MEIS2 | 15 | 35159903 | T | C | 432 | 196 | 398 | 230 | 0.04272 | 0.04914 | 156 | 120 | 38 | 121 | 156 | 37 | 0.010403 | 1 | 0.006238 |
| rs7161976 | MEIS2 | 15 | 35161477 | C | A | 595 | 33 | 573 | 55 | 0.01502 | 0.01981 | 281 | 33 | 0 | 263 | 47 | 4 | 0.029517 | 0.123805 | 0.045783 |
| rs1464284 | MEIS2 | 15 | 35173799 | T | G | 345 | 283 | 317 | 311 | 0.11355 | 0.12699 | 105 | 135 | 74 | 76 | 165 | 73 | 0.021783 | 1 | 0.013511 |
| rs1800588 | LIPC | 15 | 56510967 | C | T | 481 | 147 | 515 | 113 | 0.01789 | 0.02143 | 183 | 115 | 16 | 211 | 93 | 10 | 0.057804 | 0.316643 | 0.025756 |
| rs261332 | LIPC | 15 | 56514617 | G | A | 466 | 136 | 499 | 105 | 0.02375 | 0.0256 | 178 | 110 | 13 | 206 | 87 | 9 | 0.065462 | 0.395642 | 0.022328 |
| rs7183248 | LIPC | 15 | 56586337 | T | A | 495 | 133 | 465 | 163 | 0.0461 | 0.05374 | 202 | 91 | 21 | 175 | 115 | 24 | 0.085017 | 0.757344 | 0.03407 |
| rs2899631 | LIPC | 15 | 56589377 | C | G | 495 | 133 | 464 | 164 | 0.03953 | 0.04624 | 202 | 91 | 21 | 175 | 114 | 25 | 0.087949 | 0.646317 | 0.03407 |
| rs3829460 | LIPC | 15 | 56645237 | A | T | 438 | 188 | 473 | 155 | 0.03357 | 0.03656 | 158 | 122 | 33 | 184 | 105 | 25 | 0.113517 | 0.273781 | 0.045068 |
| rs7180596 | CA12 | 15 | 61447672 | G | A | 615 | 5 | 626 | 0 | 0.02436 | 0.03026 | 305 | 5 | 0 | 313 | 0 | 0 | 0.078503 | 1 | 0.030011 |
| rs951266 | CHRNA5 | 15 | 76665596 | C | T | 439 | 189 | 401 | 227 | 0.02271 | 0.02648 | 154 | 131 | 29 | 133 | 135 | 46 | 0.065544 | 0.048408 | 0.109056 |
| rs16969968 | CHRNA5 | 15 | 76669980 | G | A | 440 | 188 | 399 | 227 | 0.0173 | 0.01923 | 155 | 130 | 29 | 133 | 133 | 47 | 0.050379 | 0.028021 | 0.092535 |
| MRD_3767 | ENSG00000185232 | 15 | 89256462 | C | T | 558 | 30 | 600 | 12 | 0.00308 | 0.00416 | 265 | 28 | 1 | 294 | 12 | 0 | 0.013115 | 0.49 | 0.005379 |
| rs35181 | CDH11 | 16 | 63598622 | G | T | 320 | 308 | 345 | 283 | 0.15757 | 0.17483 | 86 | 148 | 80 | 89 | 167 | 58 | 0.095145 | 0.042751 | 0.858752 |
| rs1520231 | CDH11 | 16 | 63634145 | T | C | 338 | 290 | 343 | 285 | 0.77704 | 0.82079 | 100 | 138 | 76 | 87 | 169 | 58 | 0.039717 | 0.09753 | 0.295007 |
| rs8048351 | CDH11 | 16 | 63660793 | C | A | 342 | 286 | 340 | 286 | 0.95875 | 1 | 108 | 126 | 80 | 92 | 156 | 65 | 0.049251 | 0.18482 | 0.198802 |
| rs2877427 | CDH11 | 16 | 63699839 | A | G | 517 | 111 | 482 | 146 | 0.01436 | 0.01729 | 216 | 85 | 13 | 179 | 124 | 11 | 0.004275 | 0.835651 | 0.002904 |
| rs3803704 | ENSG00000180917 | 16 | 69876078 | A | G | 482 | 146 | 458 | 170 | 0.11861 | 0.13468 | 192 | 98 | 24 | 164 | 130 | 20 | 0.029347 | 0.639522 | 0.029591 |
| rs11654176 | NALP1 | 17 | 5398562 | T | C | 533 | 95 | 511 | 117 | 0.09746 | 0.11351 | 222 | 89 | 3 | 212 | 87 | 15 | 0.016138 | 0.006742 | 0.437036 |
| rs3744717 | NALP1 | 17 | 5402238 | T | C | 566 | 62 | 548 | 80 | 0.10873 | 0.12959 | 253 | 60 | 1 | 243 | 62 | 9 | 0.036254 | 0.020513 | 0.378123 |
| rs7214862 | COX10 | 17 | 13941530 | T | G | 540 | 88 | 511 | 117 | 0.02682 | 0.03235 | 228 | 84 | 2 | 207 | 97 | 10 | 0.026241 | 0.036788 | 0.083534 |
| rs1860084 | COX10 | 17 | 13950591 | T | C | 527 | 99 | 493 | 133 | 0.0134 | 0.01626 | 215 | 97 | 1 | 194 | 105 | 14 | 0.00178 | 8.59E−04 | 0.092909 |
| rs12150592 | FBXW10 | 17 | 18620558 | A | G | 565 | 63 | 580 | 48 | 0.13592 | 0.16376 | 257 | 51 | 6 | 266 | 48 | 0 | 0.04403 | 0.030506 | 0.392345 |
| rs8081248 | NOS2A | 17 | 23106091 | G | A | 340 | 240 | 373 | 247 | 0.58706 | 0.59693 | 90 | 160 | 40 | 118 | 137 | 55 | 0.026514 | 0.218328 | 0.072259 |
| rs2715553 | RARA | 17 | 35749846 | T | C | 308 | 300 | 342 | 280 | 0.12862 | 0.13756 | 78 | 152 | 74 | 104 | 134 | 73 | 0.091864 | 0.850124 | 0.042005 |
| rs7220606 | ITGB3 | 17 | 42729794 | G | A | 367 | 241 | 347 | 275 | 0.1041 | 0.10608 | 120 | 127 | 57 | 95 | 157 | 59 | 0.049007 | 1 | 0.022448 |
| rs11650072 | ITGB3 | 17 | 42748664 | C | T | 389 | 177 | 383 | 189 | 0.52276 | 0.52641 | 141 | 107 | 35 | 123 | 137 | 26 | 0.044422 | 0.224333 | 0.110601 |
| rs2696247 | COL1A1 | 17 | 45624902 | A | G | 414 | 52 | 457 | 85 | 0.03665 | 0.04237 | 186 | 42 | 5 | 189 | 79 | 3 | 0.010975 | 0.480519 | 0.010521 |
| rs8905 | PRKAR1B | 17 | 64039397 | T | G | 544 | 84 | 571 | 57 | 0.01581 | 0.01987 | 235 | 74 | 5 | 261 | 49 | 4 | 0.037717 | 1 | 0.014151 |
| rs16952176 | PTPRM | 18 | 7582299 | A | G | 627 | 1 | 620 | 8 | 0.01919 | 0.03839 | 313 | 1 | 0 | 306 | 8 | 0 | 0.063178 | 1 | 0.037724 |
| rs4130485 | PTPRM | 18 | 8251221 | G | T | 412 | 216 | 387 | 241 | 0.14258 | 0.15922 | 130 | 152 | 32 | 125 | 137 | 52 | 0.059651 | 0.025478 | 0.745197 |
| rs6417047 | PTPRM | 18 | 8264246 | G | A | 344 | 284 | 307 | 321 | 0.03667 | 0.04201 | 92 | 160 | 62 | 84 | 139 | 91 | 0.025537 | 0.009115 | 0.534044 |
| rs582420 | PTPRM | 18 | 8322974 | G | A | 459 | 163 | 431 | 195 | 0.05349 | 0.06037 | 164 | 131 | 16 | 154 | 123 | 36 | 0.016144 | 0.00549 | 0.37987 |
| rs12456630 | PTPRM | 18 | 8327540 | A | G | 366 | 260 | 403 | 225 | 0.03805 | 0.04238 | 113 | 140 | 60 | 135 | 133 | 46 | 0.136793 | 0.137044 | 0.086382 |
| rs609039 | PTPRM | 18 | 8393289 | A | C | 454 | 170 | 488 | 140 | 0.04244 | 0.04939 | 161 | 132 | 19 | 190 | 108 | 16 | 0.080186 | 0.606565 | 0.0296 |
| rs8086835 | NEDD4L | 18 | 54132690 | T | C | 434 | 190 | 423 | 205 | 0.40344 | 0.42925 | 143 | 148 | 21 | 146 | 131 | 37 | 0.06475 | 0.037967 | 0.872972 |
| rs4499341 | INSR | 19 | 7151990 | T | C | 375 | 253 | 411 | 217 | 0.03581 | 0.04122 | 109 | 157 | 48 | 131 | 149 | 34 | 0.099457 | 0.123253 | 0.084507 |
| rs8108661 | INSR | 19 | 7155619 | C | A | 302 | 326 | 330 | 298 | 0.11407 | 0.12754 | 64 | 174 | 76 | 88 | 154 | 72 | 0.077417 | 0.777962 | 0.031927 |
| rs890860 | INSR | 19 | 7179165 | C | T | 510 | 118 | 498 | 128 | 0.45989 | 0.47746 | 199 | 112 | 3 | 198 | 102 | 13 | 0.034766 | 0.011593 | 1 |
| rs3745364 | STXBP2 | 19 | 7605205 | C | T | 385 | 227 | 427 | 199 | 0.0496 | 0.05551 | 126 | 133 | 47 | 151 | 125 | 37 | 0.163969 | 0.240396 | 0.089514 |
| rs8104576 | LDLR | 19 | 11063569 | G | A | 619 | 5 | 628 | 0 | 0.02459 | 0.03051 | 307 | 5 | 0 | 314 | 0 | 0 | 0.07916 | 1 | 0.03026 |
| rs10417567 | NOTCH3 | 19 | 15169068 | C | T | 538 | 60 | 533 | 87 | 0.03222 | 0.03476 | 241 | 56 | 2 | 228 | 77 | 5 | 0.092333 | 0.450955 | 0.043129 |
| rs10854166 | FKBP8 | 19 | 18513844 | T | C | 339 | 273 | 324 | 300 | 0.2214 | 0.23119 | 100 | 139 | 67 | 77 | 170 | 65 | 0.048042 | 0.769036 | 0.032609 |
| rs851303 | LIPE | 19 | 47599284 | T | A | 479 | 97 | 482 | 110 | 0.43602 | 0.44448 | 194 | 91 | 3 | 198 | 86 | 12 | 0.064781 | 0.033139 | 0.929925 |
| rs2694555 | DHX34 | 19 | 52577355 | A | C | 587 | 39 | 605 | 23 | 0.03599 | 0.03769 | 278 | 31 | 4 | 291 | 23 | 0 | 0.06455 | 0.061506 | 0.100074 |
| rs1042265 | FTL | 19 | 54163632 | C | T | 481 | 71 | 533 | 53 | 0.03885 | 0.04538 | 209 | 63 | 4 | 242 | 49 | 2 | 0.114908 | 0.437981 | 0.049341 |
| rs3219281 | NR1H2 | 19 | 55578899 | C | T | 564 | 50 | 546 | 74 | 0.02673 | 0.02935 | 258 | 48 | 1 | 244 | 58 | 8 | 0.033982 | 0.03772 | 0.098384 |
| rs241606 | RNF24-FTLL1 | 20 | 3863885 | T | G | 373 | 255 | 334 | 294 | 0.02652 | 0.0306 | 114 | 145 | 55 | 94 | 146 | 74 | 0.094186 | 0.075159 | 0.107083 |
| rs17287269 | RNF24-FTLL1 | 20 | 3890178 | A | C | 308 | 320 | 348 | 280 | 0.02385 | 0.02755 | 81 | 146 | 87 | 100 | 148 | 66 | 0.086707 | 0.062796 | 0.112641 |
| rs6116130 | RNF24-FTLL1 | 20 | 3895026 | A | T | 540 | 86 | 513 | 115 | 0.02728 | 0.03101 | 234 | 72 | 7 | 207 | 99 | 8 | 0.050255 | 1 | 0.018158 |
| rs998132 | RNF24-FTLL1 | 20 | 3913054 | C | T | 310 | 318 | 349 | 279 | 0.02755 | 0.03175 | 82 | 146 | 86 | 100 | 149 | 65 | 0.09389 | 0.061618 | 0.134721 |
| rs17287822 | RNF24-FTLL1 | 20 | 3926536 | C | T | 541 | 87 | 513 | 115 | 0.03151 | 0.03791 | 234 | 73 | 7 | 207 | 99 | 8 | 0.05931 | 1 | 0.02314 |
| rs6114735 | C20orf39 | 20 | 24393155 | A | G | 418 | 210 | 450 | 176 | 0.04111 | 0.04358 | 139 | 140 | 35 | 163 | 124 | 26 | 0.122257 | 0.280966 | 0.055204 |
| rs4815272 | C20orf39 | 20 | 24396895 | T | A | 408 | 212 | 459 | 163 | 0.00217 | 0.00244 | 132 | 144 | 34 | 172 | 115 | 24 | 0.005998 | 0.170761 | 0.001736 |
| rs6049737 | C20orf39 | 20 | 24420839 | C | G | 349 | 269 | 387 | 239 | 0.05499 | 0.05714 | 94 | 161 | 54 | 122 | 143 | 48 | 0.081153 | 0.516275 | 0.028516 |
| rs6138332 | C20orf39 | 20 | 24512317 | A | G | 331 | 257 | 329 | 289 | 0.28651 | 0.29786 | 83 | 165 | 46 | 88 | 153 | 68 | 0.106764 | 0.048598 | 1 |
| rs4813522 | C20orf39 | 20 | 24525509 | T | C | 529 | 99 | 544 | 84 | 0.23027 | 0.26281 | 228 | 73 | 13 | 232 | 80 | 2 | 0.014834 | 0.006737 | 0.786878 |
| rs1736493 | PLTP | 20 | 43964041 | T | C | 577 | 49 | 598 | 30 | 0.02622 | 0.02746 | 267 | 43 | 3 | 284 | 30 | 0 | 0.053988 | 0.123805 | 0.051035 |
| rs6015591 | SYCP2 | 20 | 57879024 | G | C | 384 | 228 | 354 | 258 | 0.07968 | 0.0902 | 131 | 122 | 53 | 104 | 146 | 56 | 0.069462 | 0.832733 | 0.030597 |
| rs3787751 | HLCS | 21 | 37082033 | C | T | 348 | 264 | 318 | 304 | 0.0432 | 0.04568 | 98 | 152 | 56 | 84 | 150 | 77 | 0.112718 | 0.062608 | 0.185813 |
| rs8132225 | HLCS | 21 | 37085192 | C | T | 571 | 53 | 544 | 82 | 0.00872 | 0.01052 | 260 | 51 | 1 | 240 | 64 | 9 | 0.013115 | 0.02051 | 0.045262 |
| rs732252 | HLCS | 21 | 37087251 | C | T | 582 | 44 | 599 | 29 | 0.06829 | 0.0714 | 270 | 42 | 1 | 289 | 21 | 4 | 0.008896 | 0.373002 | 0.021088 |
| rs3787755 | HLCS | 21 | 37095567 | C | G | 412 | 216 | 444 | 184 | 0.05261 | 0.06037 | 131 | 150 | 33 | 163 | 118 | 33 | 0.025941 | 1 | 0.013108 |
| rs1041832 | HLCS | 21 | 37104999 | A | G | 352 | 276 | 388 | 240 | 0.03895 | 0.04466 | 90 | 172 | 52 | 123 | 142 | 49 | 0.017703 | 0.828116 | 0.006923 |
| rs2835535 | HLCS | 21 | 37222089 | G | A | 337 | 285 | 380 | 246 | 0.01979 | 0.02199 | 87 | 163 | 61 | 112 | 156 | 45 | 0.057756 | 0.088527 | 0.039413 |
| rs3827189 | HLCS | 21 | 37233863 | T | C | 321 | 307 | 357 | 271 | 0.04154 | 0.04183 | 75 | 171 | 68 | 97 | 163 | 54 | 0.099653 | 0.18965 | 0.060055 |
| rs762376 | HLCS | 21 | 37262526 | C | T | 360 | 264 | 333 | 295 | 0.09675 | 0.09952 | 106 | 148 | 58 | 83 | 167 | 64 | 0.12041 | 0.614304 | 0.045222 |
| rs915845 | ABCG1 | 21 | 42508507 | T | G | 546 | 78 | 523 | 105 | 0.03458 | 0.03752 | 238 | 70 | 4 | 219 | 85 | 10 | 0.090417 | 0.174686 | 0.071926 |
| rs178744 | ABCG1 | 21 | 42535865 | G | A | 434 | 194 | 397 | 231 | 0.02735 | 0.03174 | 154 | 126 | 34 | 122 | 153 | 39 | 0.035695 | 0.618725 | 0.012624 |
| rs2839478 | ABCG1 | 21 | 42540151 | G | A | 430 | 198 | 404 | 224 | 0.12037 | 0.13527 | 151 | 128 | 35 | 124 | 156 | 34 | 0.066339 | 1 | 0.036419 |
| rs225398 | ABCG1 | 21 | 42561663 | G | C | 290 | 200 | 254 | 206 | 0.21684 | 0.23758 | 73 | 144 | 28 | 49 | 156 | 25 | 0.086193 | 0.884809 | 0.03608 |
| rs3788010 | ABCG1 | 21 | 42589091 | A | G | 348 | 268 | 389 | 237 | 0.04279 | 0.04344 | 91 | 166 | 51 | 120 | 149 | 44 | 0.067911 | 0.435441 | 0.02228 |
| rs1541290 | ABCG1 | 21 | 42591552 | A | G | 315 | 311 | 355 | 273 | 0.02753 | 0.03142 | 74 | 167 | 72 | 99 | 157 | 58 | 0.066289 | 0.168988 | 0.031893 |
| rs2839483 | ABCG1 | 21 | 42594106 | A | G | 384 | 240 | 422 | 204 | 0.03003 | 0.03334 | 111 | 162 | 39 | 138 | 146 | 29 | 0.073245 | 0.201604 | 0.03364 |
| rs738809 | ENSG00000099991 | 22 | 22730046 | A | G | 420 | 196 | 450 | 176 | 0.15428 | 0.17287 | 151 | 118 | 39 | 157 | 136 | 20 | 0.023857 | 0.009026 | 0.809882 |
| rs13057834 | ENSG00000099991 | 22 | 22863646 | T | C | 543 | 19 | 607 | 9 | 0.03074 | 0.03522 | 262 | 19 | 0 | 299 | 9 | 0 | 0.091443 | 1 | 0.033089 |
| rs1009544 | SREBF2 | 22 | 40563848 | C | G | 539 | 89 | 535 | 91 | 0.85386 | 0.87228 | 228 | 83 | 3 | 233 | 69 | 11 | 0.051986 | 0.033051 | 0.651017 |
| rs133287 | SREBF2 | 22 | 40590919 | G | A | 277 | 283 | 310 | 248 | 0.04142 | 0.04201 | 60 | 157 | 63 | 83 | 144 | 52 | 0.070258 | 0.295506 | 0.025966 |
| rs4253755 | PPARA | 22 | 44935895 | G | A | 541 | 79 | 555 | 69 | 0.35892 | 0.38172 | 238 | 65 | 7 | 243 | 69 | 0 | 0.027806 | 0.00738 | 0.774255 |
| rs6007662 | PPARA | 22 | 44941564 | A | G | 470 | 158 | 479 | 149 | 0.55457 | 0.59944 | 182 | 106 | 26 | 178 | 123 | 13 | 0.059612 | 0.046003 | 0.808777 |
| rs4253760 | PPARA | 22 | 44942903 | T | G | 511 | 117 | 515 | 113 | 0.77042 | 0.82679 | 216 | 79 | 19 | 208 | 99 | 7 | 0.018906 | 0.025756 | 0.550923 |
| rs1042311 | PPARA | 22 | 44948299 | C | T | 619 | 7 | 627 | 1 | 0.03295 | 0.03824 | 306 | 7 | 0 | 313 | 1 | 0 | 0.101389 | 1 | 0.037724 |

TABLE 2

Sequence Definitions of Polymorphic Sites
Discovered by MRD

MRD_3565 SEQ ID NO 3:
cctaccaggcagaggtgtttaaccccttcgttggcgagccagctcctcca
ggacacagccatgccgccggccccgccgcgctgcgctctgtgcccgccgg
Mccgagaaggagcgggcggcggccggggcagcggttacagttgtgcggcc
tgccgggccgctgagcgaggaccgagggtcaaagactgagtggaagcccg
a
MRD_3435 SEQ ID NO 4:
Cgtcctgtgggtcttgagcagcagacagtttctttctgcctggacccccg
cccccaccccaaaagaggccacagagcttcagcaggaagtttggcctccc
Ygcccgtctccagggaagcagcttttggtccccatctggggcaagcctcc
atgcccaaacatggtcaagtctgagcacacagcctggagacacagactcg
g
MRD_3567 SEQ ID NO 5
ggccttaccttcgcgcctgcagccgcaggtcctgctctgaggggctgaac
acatgctggagctggtgcttggcaattgcctgccacttgcctctgttttc
Wcgctccagccgctcccagatttctgggatctaggagagagaagtggaga
gtggcaggaaggtgctggtaaagtgggacagtggtcctgagcagctaact
t
MRD_3459 SEQ ID NO 6
gaaaaccctccagtcagcgcttatcccttctgctctctcccctcacccag
agaaatacatggagtttgaccttaatggaaatggcgatattggtgagaaa
Ygggtgatttgcgggggcagggtggtgtgcaggcctaagaagacagaggt
ctctcctacatgctccattcctcatgatttgggagggggcccacctacca
c
MRD_3427 SEQ ID NO 7
aaaattaggagtggaattactgagtcatagggtatgcatgtgttcaactt
ttgtggatgtttccacatgattttccaaagtgaaaccctgttttgagatc
Kggaatattgactgtctttcactctccttggaccctagggctacagaact
ggctacccttatcgataccctgctctgagagagaagtctaaaaaatacag
c
MRD_3470 SEQ ID NO 8
gctctaattcatgcaattaacgccttctgtatgaaacagtttttcctcct
ttctttaagggggtggggatacaaaagtaactcagaaaattttctttgtc
Mtaaaactacactgaacatgtgaatagcatattgtggtggacaagagcaa
gagtaaacagatgaaaagaataaatgtttagatttgttgataaaacagga
a

What is claimed is:

1. A method of identifying a patient having an increased susceptibility to CAD, the method comprising the steps of:
obtaining a sample of DNA or RNA from the patient; and
determining which alleles are present in the sample at one or more at-risk polymorphisms selected from the group listed in Table 1; and
identifying the patient as having an increased susceptibility to CAD if the at risk allele is present in the patient.

2. The method of claim 1 wherein said one or more at-risk polymorphism is located in PARD3 or CDC42.

3. The method of claim 2 wherein at least one at-risk polymorphism is located in PARD3 and at least one at-risk polymorphism is located in CDC42.

4. The method of claim 1, wherein the patient is human.

5. The method of claim 3 wherein said at least one at-risk polymorphism in CDC42 is rs16826506 and wherein said at least one at-risk polymorphism in PARD3 is rs1545214.

6. The method of claim 3 wherein a C at rs16826506 and an A at rs1545214 indicate that the patient has an increased susceptibility to CAD.

7. A method for determining if a patient has an increased susceptibility to CAD, the method comprising the steps of:
obtaining a nucleic acid sample from said patient;
determining if a C is present at SNP rs16826506;
determining if an A is present at SNP rs1545214; and determining that the patient has an increased susceptibility to CAD if a C is present at SNP rs16826506 and an A is present at SNP rs1545214.

8. The method of claim 7 wherein the patient is human.

9. The method of claim 7 wherein the sample is obtained from blood or saliva.

10. A method for identifying a patient as having an increased susceptibility to CAD, the method comprising the steps of:
obtaining a nucleic acid sample from said patient;
determining if a C is present at position 31 of SEQ ID NO 1 and an A at position 31 of SEQ ID NO 2; and
identifying the patient as having an increased susceptibility to CAD if the patient has a C is present at position 31 of SEQ ID NO 1 and an A at position 31 of SEQ ID NO 2.

11. The method of claim 10 where the patient is human.

12. The method of claim 10 where the sample is obtained from blood or saliva.

13. The method of claim 10 wherein the step of determining is carried out by allele specific hybridization.

14. The method of claim 10 wherein the step of determining is carried out by allele specific primer extension.

15. The method of claim 10 wherein the step of determining is carried out by molecular inversion probe analysis.

16. The method of claim 10 wherein the step of determining is carried out by oligonucleotide ligation assay.
